(19) United States
(12) Patent Application Publication
(10) Pub. No.: US 2007/0185323 A1
(43) Pub. Date: Aug. 9, 2007

(54) Method of preparing benzazepines and derivatives thereof

(76) Inventors: Samir Zard, Gif-Sur-Yvette (FR); Alejandro Cordero Vargas, Paris (FR); Beatrice Sire, Palaiseau (FR)

Correspondence Address: OLIFF & BERRIDGE, PLC, P.O. BOX 19928, ALEXANDRIA, VA, 22320, US

(21) Appl. No.: 10/591,175

(22) PCT Filed: Feb. 21, 2005
(86) PCT No.: PCT/FR05/50110

§ 371 Date: Dec. 14, 2006

(30) Foreign Application Priority Data
Mar. 2, 2004 (FR) 04 50416

(51) Int. Cl. C07D 223/16; C07C 251/32

(52) U.S. Cl. 540/593; 564/253

(57) ABSTRACT

The invention relates to a method of preparing benzazepine compounds having general formula (IA) consisting in reacting at least one compound having general formula (IIA) with an olefin, the compound thus obtained then being cyclised such as to produce tetralone, followed by the oxime derivative of same, which, by transformation by a Beckmann rearrangement, gives rise to the desired compounds.

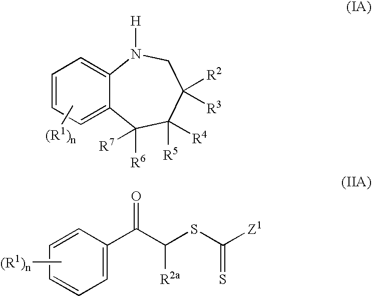

The present invention is aimed more particularly at providing a novel route of access to benzazepine-type molecules.

Benzazepines and related molecules such as benzazepinones and benzodiazepines constitute families of compounds which are advantageous for their pharmacological activities.

Recently, it has thus been shown that two N-substituted benzazepine derivatives, i.e. (±)-N-[4-(7-chloro-5-hydroxy-2,3,4,5-tetrahydro-1H-1-benz-azepin-1-ylcarbonyl)-3-methylphenyl]-2-methylbenzamide (OPC-41061) and (±)-N-[4-(7-chloro-5-hydroxy-2,3,4,5-tetrahydro-1H-1-benzazepin-1-ylcarbonyl)-phenyl]-2-methylbenzamide (OPC-31260) from the company OTSUKA PHARMACEUTICALS, can act as powerful arginine vasopressin (AVP) V₂ receptor antagonists and therefore be effectively used for the treatment of cardiac disorders.

However, the various methods of synthesis currently available for obtaining these benzazepine derivatives constitute a major obstacle, firstly, to obtaining these compounds under satisfactory conditions in terms of yield and cost and, secondly, to the development of novel derivatives.

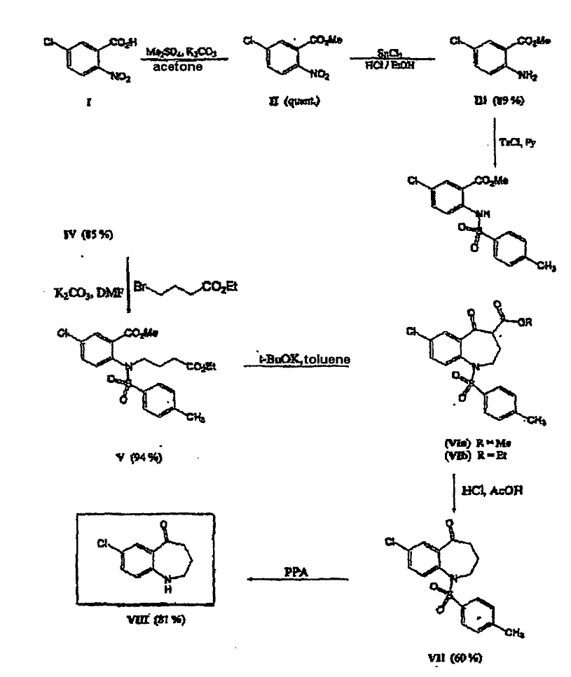

Thus, the method, represented in FIG. 1, which corresponds to the synthetic pathway currently used to obtain the OPC-41061 derivative comprises eleven consecutive stages, some of which involve drastic conditions which are not compatible with the presence of a certain number of functional groups.

A subject of the present invention is precisely to provide a novel route of access to benzazepine-type compounds, which advantageously makes it possible, firstly, to prepare known compounds under satisfactory conditions and, secondly, to gain access to novel derivatives of these compounds.

More specifically, according to a first of its aspects, the present invention relates to a method of preparing at least one benzazepine compound of general formula (IA): 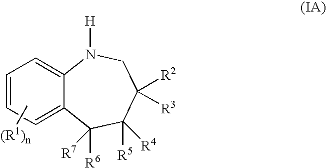
in which:
R¹ represents a halogen atom chosen from chlorine, fluorine, bromine and iodine, an alkyl, haloalkyl, alkenyl, alkynyl, acyl, aryl, arylalkyl, arylalkenyl or arylalkynyl group, or else a hydrocarbon-based ring or a heterocycle, a polymer chain, or a group —(CH₂)ₘ—R^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, or —(CH₂)ₘ—NR^{k}R¹, with:
R^{k}, R¹ and R^{m} each independently denoting a hydrogen atom, an alkyl, haloalkyl, acyl, aryl, alkenyl, arylalkenyl, alkynyl, arylalkynyl, aralkyl or alkaryl group, a hydrocarbon-based ring or a heterocycle, or else R^{k} and R¹ form, together with the atom to which they are attached, a heterocycle,
with m denoting an integer greater than or equal to 0, especially ranging from 0 to 100, and in particular ranging from 0 to 20,
n represents an integer chosen from 0, 1, 2, 3 and 4, with, when n is greater than or equal to 2, it being possible for the corresponding R¹ groups to be identical or different, and, where appropriate, to form, together, a hydrocarbon-based ring or a heterocycle, for example with 5 or 6 ring members,
R², R³, R⁴, R⁵, R⁶ and R⁷ represent, independently of one another, a hydrogen atom, a halogen atom chosen from chlorine, fluorine and bromine, an alkyl, haloalkyl, alkenyl, alkynyl, acyl, aryl, arylalkyl, arylalkenyl or arylalkynyl group, or else a hydrocarbon-based ring or a heterocycle, a polymer chain, or a group —(CH₂)ₘ—R^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘSiR^{k}R¹R^{m}, —(CH₂)ₘ—COOR^{k}, —(CH₂)ₘ—NCOR^{k} or —(CH₂)ₘ—NR^{k}R¹, with R^{k}, R¹, R^{m} and m as defined above,
   or R⁴, R⁵, R⁶ and R⁷ form, in pairs, one or more hydrocarbon-based ring(s) or heterocycle(s), with at least one of the R⁴, R⁵, R⁶ and R⁷ groups representing a hydrogen atom,
   from at least one compound of general formula (IIA) 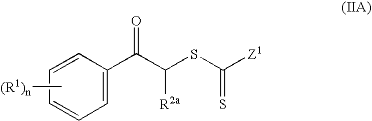
   in which
Z¹ represents a group chosen from:
(i) alkyl, acyl, aryl, aralkyl, alkene or alkyne groups, and hydrocarbon-based rings or heterocycles,
(ii) an —OR^{a} or —SR² group in which R^{a} is a group chosen from:
   an alkyl, haloalkyl, alkenyl, alkynyl, acyl, aryl, arylalkyl, arylalkenyl or arylalkynyl group, or else a hydrocarbon-based ring or a heterocycle, or else a polymer chain;
   a —CR^{b}R^{c}PO(OR^{d})(OR^{e}) group in which:
      R^{b} and R^{c} each represent, independently of one another, a hydrogen atom, a halogen atom, an alkyl or perfluoroalkyl group, a hydrocarbon-based ring or a heterocycle, or else an —NO₂, —NCO or —CN group, or a group chosen from the groups of type —R^{f}, —SO₃R^{f}, —OR^{f}, —SR^{f}, —NR^{f}R^{g}, —COOR^{f}, —O₂CR^{f}, —CONR^{f}R^{g}, —NR^{f}COR^{g}, in which R^{f} and R^{g} each independently denote an alkyl, alkenyl, alkynyl, cycloalkenyl, cycloalkynyl or aryl group optionally condensed with a heterocycle, alkaryl, arylalkyl or heteroaryl,
      or else R^{b} and R^{c} form, together with the carbon atom to which they are attached, a C═O or C═S group or else a hydrocarbon-based ring or a heterocycle; and
      R^{d} and R^{e} each represent, independently of one another, a radical corresponding to one of the definitions given above for the R^{f} group;
      or else R^{d} and R^{e} form, together, a hydrocarbon-based chain containing from 2 to 4 carbon atoms, optionally interrupted with a group chosen from —O—, —S— and —NR^{h}; where R^{h} corresponds to one of the definitions given above for the R^{f} group;
(iii) an —NRⁱR^{j} group, in which:
   R^{j} and R^{j} represent, independently of one another, a radical chosen from an alkyl, haloalkyl, alkenyl, alkynyl, acyl, ester, aryl, arylalkyl, arylalkenyl or arylalkynyl group, or else a hydrocarbon-based ring or a heterocycle; or
   R^{j} and R^{j} form, together, a hydrocarbon-based chain containing from 2 to 4 carbon atoms, optionally interrupted with an —O—, —S—, or —NR^{h}— group, where R^{h} corresponds to one of the definitions given above for the R^{f} group (said hydrocarbon-based chain advantageously forming a 5-membered ring with the nitrogen atom to which Rⁱ and R^{j} are attached),
R^{2a} represents a group chosen from a hydrogen atom, a halogen atom, in particular fluorine, chlorine or bromine, an alkyl, haloalkyl, acyl, aryl or arylalkyl group, or else a hydrocarbon-based ring or a heterocycle, a polymer chain, or a group —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR₁), (CH₂)ₘ—SiR^{k}R¹R^{m}, —(CH₂)ₘ—COOR^{k}, —(CH₂)ₘ—NCOR^{k} or —(CH₂)ₘ—NR^{k}R¹, in which R^{k}, R¹, R^{m} and m are as defined above, and preferably a hydrogen atom,
R¹ and n are as defined above,
   comprising at least the stages consisting in:
a—reacting said compound of general formula (IIA) with at least one olefin of general formula (A) 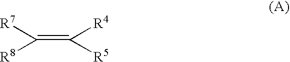
   in which:
R⁴, R⁵, R⁶ and R⁷ are as defined above, with at least one of the R⁴, R⁵, R⁶ or R⁷ groups representing a hydrogen atom,
so as to obtain at least one compound of general formula (IIIA) 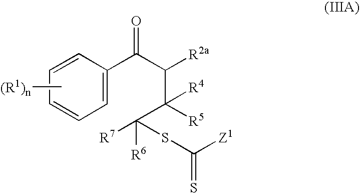
   in which:
R¹, R^{2a}, R⁴, R⁵, R⁶, R⁷, Z¹ and n are as defined above,
   b—cyclizing, by radical-based process, said compound of general formula (IIIA) so as to obtain at least one tetralone compound of general formula (IVA) 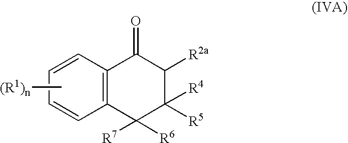
      in which:
R¹, R^{2a}, R⁴, R⁵, R⁶, R⁷ and n are as defined above,
   c—converting said compound of general formula (IVA) into at least its oxime derivative of general formula (VA) 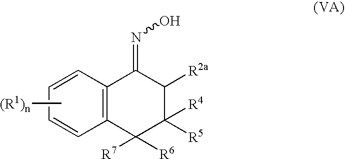
      in which:
R¹, R^{2a}, R⁴, R⁵, R⁶, R⁷ and n are as defined above,
   d—converting said compound of general formula (VA), by Beckmann rearrangement and consecutive reduction(s), into at least one compound of general formula (IA), and
   e—recovering said compound of general formula (IA).

According to another of its aspects, a subject of the invention is also a method of preparing at least one compound of general formula (1B) 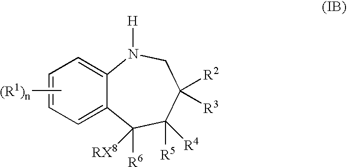
in which:
R¹, R², R³, R⁴, R⁵, R⁶ and n are as defined above,
X represents O, NR⁹, S, S(O), SO₂, SO₂NR⁹, and
R⁸ and R⁹ represent, independently of one another, a hydrogen atom, an alkyl, haloalkyl, alkenyl, alkynyl, acyl, aryl, arylalkyl, alkaryl, arylalkenyl or arylalkynyl group, or else a hydrocarbon-based ring or a heterocycle, or a polymer chain, where appropriate substituted,
   or else R⁸ and R⁹ form, together with the atom to which they are attached, a heterocycle,
   from at least one compound of general formula (IVB) 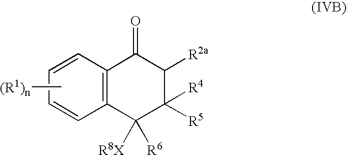
   in which:
R¹, R⁴, R⁵, R⁶, R⁸, X and n are as defined above, and
R^{2a} is as defined above,
   comprising at least the stages consisting in:
   a′—converting said compound of general formula (IVB) into at least its oxime derivative of general formula (VB) 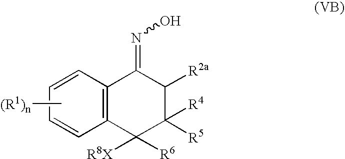
      in which:
R¹, R^{2a}, R⁴, R⁵, R⁶, R⁸, X and n are as defined above,
   b′—converting said compound of general formula (VB), by Beckmann rearrangement and consecutive reduction(s), into at least said compound of general formula (IB), and
   c′—recovering said compound of general formula (IB).

Throughout the present description, the term “alkyl group” is intended to cover a linear or branched, saturated hydrocarbon-based group which may optionally include one or more saturated aliphatic ring(s). For the purpose of the invention, the alkyl groups can have up to 25 carbon atoms, especially from 1 to 12 carbon atoms, and in particular from 1 to 6 carbon atoms.

Among the alkyl radicals that can be envisioned, mention may in particular be made of the methyl, ethyl, propyl, isopropyl, butyl, tert-butyl, pentyl, hexyl, octyl, decyl or dodecyl radical.

In particular, an alkyl group can also denote, for the purposes of the present description, a cycloalkyl group, i.e. a cyclic saturated hydrocarbon-based radical having in particular from 3 to 10 carbon atoms.

The term “alkoxy group” denotes, for its part, for the purposes of the present description, an —OAlk radical, where Alk denotes an alkyl group as defined above.

For the purposes of the present description, the term “haloalkyl group” is intended to mean an alkyl group as defined above and substituted with at least one halogen atom, where the term “halogen atom” here denotes, as in the entire description, a fluorine, chlorine, bromine or iodine atom, in particular a fluorine or chlorine atom. The “haloalkyl” groups of the invention can thus be, for example, “perfluoroalkyl” groups, i.e., for the purposes of the invention, groups corresponding to the formula —CH₂CₙF₂ₙ₊₁, where n represents an integer ranging from 1 to 20.

The term “alkenyl group”, in the sense that it is used in the present description, is intended to denote a linear or branched, unsaturated hydrocarbon-based radical having at least one double bond C═C. The alkenyl groups of the invention can have from 2 to 25 carbon atoms, especially from 2 to 12 carbon atoms, and in particular from 2 to 6 carbon atoms.

Similarly, the term “alkynyl group” is intended to mean a linear or branched, unsaturated hydrocarbon-based radical having at least one triple bond C≡C. The alkynyl groups of the invention generally have from 2 to 25 carbon atoms, especially from 2 to 15 carbon atoms, and in particular from 2 to 6 carbon atoms.

For the purposes of the present description, the terms “ester group” and “acyl group” are intended to mean, respectively, a —C(═O)—OB, and —C(═O)—B group where B denotes a saturated or unsaturated, linear or branched hydrocarbon-based chain containing from 1 to 25 carbon atoms, which can in particular be an alkyl, alkenyl or alkynyl group as defined above.

For the purposes of the present description, a radical of “hydrocarbon-based ring” type denotes a saturated, unsaturated or aromatic cyclic group, in particular of cycloalkyl, cycloalkenyl or cycloalkynyl type, optionally substituted, and containing from 3 to 20 carbon atoms. A radical of “heterocycle” type denotes, for its part, such a carbon-based ring interrupted with at least one heteroatom chosen, for example, from N, O, S, P and Si, it being possible for said carbon-based ring to be saturated or unsaturated.

For the purposes of the present description, the term “aryl group” denotes, for its part, a monocyclic or polycyclic aromatic group generally having from 5 to 20 carbon atoms, and in particular from 6 to 10 carbon atoms. Thus, it may, for example, be a phenyl group, or else a 1- or 2-naphthyl group. According to one specific variant, an “aryl” group, for the purposes of the invention, can integrate one or more heteroatoms such as sulfur, oxygen or nitrogen. In this particular case, the term “aryl group” denotes a monocyclic or polycyclic heteroaromatic group.

For the purposes of the present description, the “arylalkyl”, “aralkenyl” and “aralkynyl” groups are, respectively, alkyl, alkenyl and alkynyl chains substituted with an aryl group as defined above.

The various radicals can optionally be interrupted with one or more heteroatoms chosen in particular from O, S, N, P and Si, or with —(C═O)—, —(C═S)—, —SO₂— or —SO— groups, or secondary or tertiary amines, and they can be substituted with groups of any type not liable to interfere with the reaction under consideration or to lead to parasitic reactions between the compounds present together, and in particular with one or more groups, which may be identical or different, chosen from alkoxycarbonyl or aryloxycarbonyl (—COOR), carboxyl (—COOH), acyloxy (—O₂CR), carbamoyl (—CONR₂), cyano (—CN), alkylcarbonyl, alkylarylcarbonyl, arylcarbonyl, arylalkylcarbonyl, phthalimido, maleimido, succinimido, amidino, guanidino, hydroxyl (—OH), amnino (—NR₂) or (—NH₂), halogen, perfluoroalkyl (CₙF₂ₙ₊₁), allyl, epoxy, alkoxy (—OR), thioalkoxy or thioaryloxy (—SR), sulfone or phosphonate groups, a silylated group, a halogen atom, groups which are hydrophilic or ionic in nature, such as alkali metal salts of carboxylic acids, alkali metal salts of sulfonic or phosphonic acids, polyalkylene oxide chains (PPO, PEO), and cationic substituents (quaternary ammonium salts), R representing an alkyl or aryl group, or a polymer chain, it being possible for said substituents to be optionally interrupted with heteroatoms. It is within the scope of those skilled in the art to choose the nature of the various groups and substituents present in the compounds used in order to prevent any unwanted side reaction.

The methods according to the invention are particularly advantageous for preparing benzazepine compounds corresponding to general formula (IA) or (IB) in which n=1, and in particular in which R¹ is in the para-position (with respect to the nitrogen atom).

The R¹ group can represent a halogen atom, in particular a fluorine, chlorine, bromine or iodine atom, or an alkoxy, in particular methoxy, group.

According to one variant of the invention, the benzazepine compound can correspond to formula (IA) or (IB) in which R² and R³ each independently represent a hydrogen atom or an alkyl group.

According to another variant of the invention, the benzazepine compound can correspond to general formula (IA) or (IB) in which R² and R³ each represent a halogen atom, and in particular a chlorine, fluorine or bromine atom.

Among the compounds of general formula (IIA) which can be used in stage a—of the method constituting the first aspect of the invention, mention may in particular be made of xanthate compounds, i.e. compounds in which Z¹ represents —OR^{a} and in particular those in which Ra represents a C₁ to C₁₂ alkyl group, and in particular an ethyl group.

As regards the olefin of formula (A), it may be monosubstituted or disubstituted.

In the case of disubstituted olefins, they may be cyclic olefins such as, for example, cyclopentene or norbomene, with, in this case, either R⁴ and R⁷ or R⁶ and R⁵ each representing a hydrogen atom, or they may be terminal disubstituted olefins, i.e. with either R⁴ and R⁵, or R⁷ and R⁶ each representing a hydrogen atom.

In this case, the benzazepines according to the invention correspond to general formula (IA) or (IB) in which at least two of the substituents R⁴, R⁵, R⁶ and R⁷, and in particular either R⁴ and R⁵, or R⁷ and R⁶, or else either R⁴ and R⁷, or R⁶ and R⁵, each represent a hydrogen atom.

According to a specific variant of the invention, the olefin is monosubstituted. Also most particularly suitable for the invention are the olefins of formula (A) in which R⁴, R⁵ and R⁶ simultaneously represent a hydrogen atom, and in particular those in which R⁷ represents an —XR⁸ group as defined above.

More particularly, the benzazepines according to the invention correspond to general formula (IA) or (IB) in which R⁴, R⁵ and R⁶ simultaneously represent a hydrogen atom.

The substituent(s) of this olefin can be chosen from —O acyl groups and groups of —(CH₂)ₚCN type, with p representing an integer ranging from 1 to 10, and in particular equal to 1.

By way of illustration of the olefins of formula (A) which can be used according to the invention, mention may in particular be made of:
vinyl pivalates, allyl cyanide, and N-vinylphthalimide.

This olefin is generally placed in the presence of the compound of formula (IIA) in stage a, in a molar ratio at least equal to 1, in particular greater than or equal to 1.5. Generally, the two compounds are placed together in a form soluble in an organic solvent.

Stages a and b are generally carried out by a radical-based process. In particular, the compounds of formula (IIA) and/or (IIIA) can be subjected to an activation of photochemical nature, in particular by exposure to light, and/or chemical nature, for example by decomposition of a peroxide, such as dilauryl peroxide, or a diazo compound (thermal decomposition) or decomposition by autooxidation with the oxygen of an organometallic compound such as triethylborane, diethylzinc or trialkylaluminum.

As an example of peroxides which are particularly suitable as a source of free radicals in the method of the invention, mention may in particular be made of diisobutyryl peroxide, cumyl peroxyneodecanoate, tert-amyl peroxyneo-decanoate, di(2-ethylhexyl) peroxydicarbonate, tert-butyl peroxyneodecanoate, dibutyl peroxydicarbonate, dicetyl peroxydicarbonate, dimyristyl peroxydicarbonate, tert-butyl peroxyneoheptanoate, tert-amyl peroxypivalate, didecanoyl peroxide, tert-amyl peroxy-2-ethylhexanoate, tert-butyl peroxyisobutyrate, 1,4-di(tert-butylperoxycarbo)cyclohexane, tert-butyl peroxy-acetate, tert-butyl peroxybenzoate, di-tert-amyl peroxide, tert-butyl cumyl peroxide, bis-tert-butyl peroxide, dicumyl peroxide, dilauroyl peroxide (DLP) or di(4-tert-butylcyclohexyl) peroxydicarbonate.

In particular, stage a can be carried out in the presence of an effective amount of at least one radical initiator, in particular dilauroyl peroxide.

Irrespective of its nature, the source of free radicals employed according to the method of the invention is used under conditions which allow the production of free radicals, which is generally carried out by thermal activation, i.e. by raising the temperature of the reaction medium, generally to a temperature of the order of ambient temperature (approximately 20° C.) to 200° C., especially from 40° C. to 180° C., in particular from 80° C. to 160° C. The production of free radicals can also be carried out at low temperature, generally at a temperature below ambient temperature, in particular from 10° C. to −78° C., using sources of free radicals sensitive to the autooxidation process with oxygen. In general, the choice of the source of free radicals depends on the temperature at which it is desired to carry out the reaction.

The amount of the source of free radicals to be introduced into the medium depends on several parameters, in particular on its effectiveness, on its method of introduction, on the purity of the reagents, on the concentration of the reaction medium, and on the effectiveness of the olefin as free-radical trap. It is within the scope of those skilled in the art to adjust the amount of source of free radicals to be introduced into the medium according to these various parameters. Generally, the initiator is added several times to the reaction medium until the compound of general formula (ILA) or (IIIA) has been completely used up.

The solvent used in stage a- and/or b- is chosen from the solvents generally used in free-radical synthesis, such as 1,2-dichloroethane, dichloromethane, benzene, toluene, trifluoromethylbenzene (trifluorotoluene), chlorobenzene, hexane, cyclohexane, heptane, octane, ethyl acetate, tert-butyl alcohol, and mixtures thereof.

The reaction is generally carried out under atmospheric pressure, at the boiling point of the solvent chosen.

In the specific case of stage b-, the radical cyclization is also generally carried out in an acidic medium. In this case, the reaction can be carried out in the presence of a catalytic amount of acid, in particular of camphorsulfonic acid.

At the end of the reaction, the expected product of general formula (IVA) or (IVB) can be isolated or directly converted in the reaction medium into a compound of general formula (VA) or (VB).

The stage consisting of formation of the oxime (VA) or (VB) can be carried out conventionally. In particular, the compound of formula (IVA) or (IVB) can be placed in the presence of an effective amount of nitromethane or of hydroxylamine, and in particular of a hydroxylamine salt, such as, for example, hydroxylamine hydrochloride.

In general, the hydroxylamine is introduced in molar excess compared with the compound of general formula (IVA) or (IVB), in particular it is present in an amount of approximately 1.3 equivalents. The reaction consisting of formation of the oxime of formula (VA) or (VB) can be carried out in various solvents, such as, for example, methanol, ethanol, pyridine, toluene, benzene, and mixtures thereof, and in particular in ethanol.

In this oxime formation reaction, when hydroxylamine salt is used, a weak base, such as, for example, sodium acetate, triethylamine, NaHCO₃, Na₂CO₃ and mixtures thereof, can be added to the solution of compound of formula (IVA) or (IVB). This weak base can be present in an amount greater than or equal to 1 equivalent relative to the compound of formula (IVA) or (IVB) and/or less than 1 equivalent relative to the hydroxylamine.

The mixture comprising at least one compound of formula (IVA) or (IVB) and hydroxylamine can be heated, and in particular brought to reflux, for example for a period ranging from 30 minutes to 3 hours.

According to a specific variant, the methods of preparation according to the invention can comprise a stage consisting of recovery of the product of formula (VA) or (VB), in particular by recrystallization.

However, the compound obtained can be not purified but used as it is in the subsequent stage.

The methods of preparation according to the invention comprise a stage consisting of conversion of the compounds (VA) or (VB) by Beckmann rearrangement, according to a conventional method, as described, for example, by Donaruma and Heldt in Org. React. (NY) 1960, 11, 1. In particular, the Beckmann rearrangement can be carried out in the presence of an effective amount of a reagent such as, for example, PCI₅, concentrated HSO₄, formic acid, liquid SO₂, HMPA, SOCl₂, silica gel, P₅O₅-methanesulfonic acid, HCl-acetic acid-acetic anhydride or polyphosphoric acid (PPA).

The PCl₅ derivative is found to be particularly advantageous in particular by virtue of its effectiveness. It is generally placed in the presence of the oxime of formula (VA) or (VB) in molar excess, especially in a molar ratio greater than 2, in particular greater than or equal to 3, and most particularly ranging from 3 to 6.

The Beckmann rearrangement can be carried out in numerous solvents such as, for example, pyridine, acetic acid, phenol, toluene, benzene, ether, methylamine, cyclohexylamine, morpholine, dioxane, tetrahydrofuran (THF), chloroform, dichloromethane, or an aqueous solution of hydrochloric acid, and in particular in dichloromethane.

According to a specific embodiment, the oxime of formula (VA) and (VB) in solution is added dropwise to the solution of PCI₅, for example at 0° C., and the reaction is pursued at ambient temperature. At the end of the reaction, the reaction mixture is neutralized, for example with a saturated aqueous NaHCO₃ solution, treated with an organic solvent, such as CH₂Cl₂, dried, filtered, and then concentrated.

The product thus obtained can be used without any other purification, in the subsequent reducing stage.

The product derived from the Beckmann rearrangement, which may or may not be isolated, can be reduced with an effective amount of at least one metal reducing agent, such as, for example, magnesium, zinc or iron, and in particular zinc.

This metal reducing agent is generally used in molar excess, and in particular approximately 6 equivalents.

This reduction can be carried out in various solvents such as, for example, acetic acid, methanol or ethanol, or mixtures thereof.

The temperature at which this reduction is carried out can in particular range from 0° C. to the boiling point of the solvent used.

The product formed at the end of this reduction can be used without any other purification, in another reducing stage, involving a treatment with an effective amount of reducing agent, especially of BH₃ and in particular of BH₃.THF, POCl₃/NaBH₄, PCl₅/NaBH₄, LiAlH₄ or diisobutylaluminum hydride (DIBAH).

This second reduction can be carried out at the reflux of the solvent, in particular at the reflux of THF.

This method of reduction involving the two types of consecutive reductions produces compounds of formula (IA) or (IB) in which R² and R³ are hydrogen atoms.

According to another variant, the reduction can be carried out in a single stage with an effective amount of NaBH₄; the NaBH₄ is generally present in molar excess relative to the Beckmann rearrangement product. In this specific embodiment, the Beckmann rearrangement and the reduction with NaBH₄ can be carried out sequentially in the same container.

In the product of formula (IA) or (IB) obtained at the end of this reaction, the R² and R³ groups are chlorine atoms.

According to another of its aspects, a subject of the present invention is also compounds of general formula (IA) 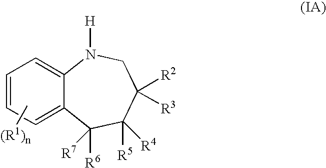
in which:

R¹, R², R³, R⁴, R⁵, R⁶, R⁷ and n are as defined above, and in particular R⁷ can represent —XR⁸, XR⁸ being as defined above.

For example, the benzazepine compound can correspond to formula (IA) in which n=1, and in particular in which R¹ is in the para-position.

According to a variant of the invention, the benzazepine compound can correspond to formula (IA) in which R² and R³ each independently represent a hydrogen atom or an alkyl group.

According to another variant of the invention, the benzazepine compound can correspond to general formula (IA) in which R² and R³ each represent a chlorine atom.

In particular, the R⁷ group can represent:
an —XR⁸ group in which X can represent an oxygen atom and R⁸ can be an acyl group such as, for example —C(═O)C(CH₃)₃, or
a —CH₂)ₚCN group in which p can represent an integer ranging from 1 to 10, and in particular 1, 2, 3 or 4.

Among the compounds of formula (IA) or (IB), mention may be made of:
7-chloro-2,3,4,5-tetrahydro-1H-benzo[b]azepin-5-yl 2,2-dimethylpropionate,
7-fluoro-2,3,4,5-tetrahydro-1H-benzo[b]azepin-5-yl 2,2-dimethylpropionate,
7-methoxy-2,3,4,5-tetrahydro-1H-benzoazepin-5-yl 2,2-dimethylpropionate,
(7-fluoro-2,3,4,5-tetrahydro-1H-benzo[b]azepin-5-yl)acetonitrile,
3,3,7-tricholoro-2,3,4,5-tetrahydro-1H-benzo[b]azepin-5-yl 2,2-dimethylpropionate, and
   derivatives thereof.

According to yet another of its aspects, a subject of the present invention is the compounds of general formula (VB) 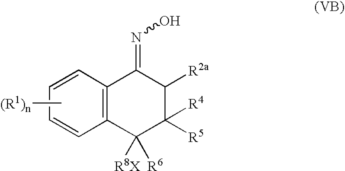
in which:

R¹, R^{2a}, R⁴, R⁵, R⁶, XR⁸ and n are as defined above.

More particularly, this compound can be chosen from
4-[(E)-hydroxyimino]-7-chloro-1,2,3,4-tetrabydronaphthalen-1-yl 2,2-dimethylpropionate,
4-[(E)-hydroxyimino]-7-fluoro-1,2,3,4-tetrahydronaphthalen-1-yl 2,2-dimethylpropionate, and
4-[(E)-hydroxyimino]-7-methoxy-1,2,3,4-tetrahydronaphthalen-1-yl 2,2-dimethylpropionate, and
   derivatives thereof.

A subject of the present invention is also a method of preparing a benzazepine of general formula (VIA): 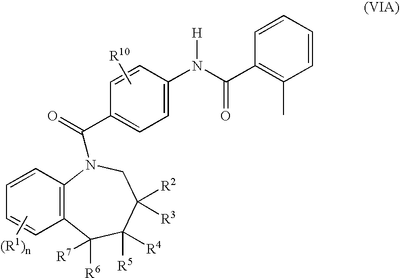
in which:
R¹, R², R³, R⁴, R⁵, R⁶, R⁷ and n are as defined above, and
R¹⁰ represents a hydrogen atom or an alkyl or acyl group, and in particular a methyl group, comprising at least the conversion of a compound of general formula (IIA) into a compound of formula (IA) according to a method in accordance with the invention.

A subject of the present invention is also a method of preparing a benzazepine of general formula (VIB): 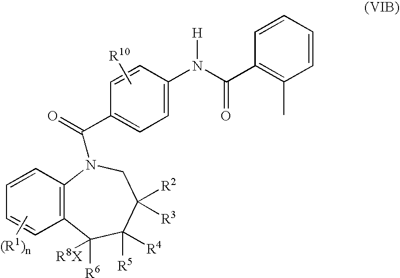
in which:
R¹, R², R³, R⁴, R⁵, R⁶, R⁸, X and n are as defined above, and
R¹⁰ represents a hydrogen atom or an alkyl or acyl group, and in particular a methyl group, comprising at least the conversion of a compound of general formula (IVB) into a compound of formula (IB), according to a method in accordance with the invention.

The following examples are given by way of nonlimiting illustration of the present invention.

### EXAMPLES

Preparation of the Compounds of General Formula (IIA)

General Method 1:

1.1 equivalents of potassium O-ethylxanthate are added, portionwise, to a solution containing 1 mmol of halogenated derivative in acetone (2 ml) at 0° C. in the dark and under argon. The solution is stirred for 1 hour at ambient temperature, and then the acetone is evaporated off under vacuum and the residue is taken up with CH₂Cl₂. The organic phase is washed with water, dried over sodium sulfate, filtered, and concentrated under vacuum. The residue obtained is purified by crystallization.

### Example 1

### 5-[2-(4-Chlorophenyl)-2-oxoethyl]-O-ethyl dithiocarbonate

According to general method 1, a solution of 20 g (85.6 mmol) of p-chlorobromoacetophenone in 172 ml of acetone at 0° C. is prepared and 15.1 g (94.2 mmol) of potassium O-ethylxanthate are added to this solution. After crystallization from water, the title product is obtained with a 96% yield in the form of yellow crystals (m.p.=64-65° C.). ¹H NMR (CDCl₃,300 MHz): 7.96 (d, 2H, CH arom, J=8.4 Hz), 7.47 (d, 2H, CH arom, J=7.5 Hz), 4.63 (q, 2H, O—CH₂, J=6.9 Hz), 4.62 (s, 2H, CO—CH₂), 1.39 (t, 3H, CH₃, J=7.1 Hz); ¹³C NMR (CDCl₃, 62.9 MHz): 213.1 (CS), 194.8 (CO), 140.3 (C—CO), 134.2 (C—Cl), 129.9 (CH arom), 129.1 (CH arom), 70.9 (CO—CH₂), 43.4 (O—CH₂), 13.8 (CH₃).

### Example 2

### 5-[2-(4-Fluorophenyl)-2-oxoethyl]-O-ethyl dithiocarbonate

According to general method 1, a solution of 20 g (115.5 mmol) of p-fluoro-y-chloroacetophenone in 232 mL of acetone at 0° C. is prepared and 20.4 g (127.4 mmol) of potassium O-ethylxanthate are added to this solution. After recrystallization with CH₂Cl₂/petroleum ether, the title product is obtained with a 98% yield in the form of yellow crystals (m.p.=58-61° C.). ¹H NMR (CDCI₃, 400 MHz): 8.07 (dd, 2H, CH arom, J=10 and 6 Hz), 7.18 (t, 2H, CH arom, J=8 Hz), 4.64 (s, 2H, CO—CH₂), 4.63 (q, 2H, O—CH₂, J=6 Hz), 1.4 (t, 3H, CH₃, J=7.1 Hz); ¹³C NMR (CDCl₃, 100.5 MHz): 213.29 (CS), 190.93 (CO), 167.43 (C—CO), 167.43; 164.88 (d, 1C, C—F, ¹J_{C—F}=256 Hz), 131.32 (CH arom), 131.23 (CH arom), 116.19 (CH arom), 115.97 (CH arom), 70.92 (CH₂—S), 43.48 (O—CH₂), 13.73 (CH₃); MS (ICP; m/z): 276 (MH⁺+NH₃), 259 (MH⁺); IR (cm⁻¹, CCl₄): 1688 (C═O), 1233 (C═S), 1052 (S—(S)C—O).

### Example 3

### 5-[2-(4-Methoxyphenyl)-2-oxoethyl]-O-ethyl dithiocarbonate

Prepared according to the protocol described in the translation Letters, 1997, 38, 1759-1762.

Preparation of the Products of General Formula (IIIA)

General Method 2:

Two equivalents of the olefin of general formula (A) are added to a solution of 1 mmol of xanthate of formula (IIA) in 1,2-dichloroethane (1 ml). The solution is brought to reflux and degassed under an argon atmosphere. After 15 minutes at reflux, 0.05 mmol of dilauroyl peroxide (DLP) is added to the reaction mixture and 0.02 mmol every 1.5 hours until the starting product has been completely used up. When the reaction is complete, the solvent is evaporated off under vacuum and the product is purified by chromatography.

### Example 4

### 1-Ethoxythiocarbonylsulfanyl-4-(4-chlorophenyl)-4-oxobutyl 2,2-dimethylpropionate

According to general method 2, a solution of 5 g (18 mmol) of xanthate of example 1 and of 5.38 ml (3.1 g, 36.3 mmol) of vinyl pivalate in 18 ml of 1,2-dichloroethane is brought to reflux and treated with DLP. The title product is obtained after silica gel chromatography (eluent: petroleum ether/ethyl acetate (95:5)) with a 97% yield (yellow oil). ¹H N (CDCl₃, 400 MHz): 7.89 (d, 2H, CH arom, J=8 Hz), 7.44 (d, 2H, CH arom, J=8 Hz), 6.71 (t, 1H, CH—S, J=8 Hz), 4.62 (dq, 2H, O—CH₂, J=8 and 4 Hz), 3.1 (dt, 2H, CO—CH₂, J=7.3 and 3.2 Hz), 2.41 (m, 2H, CH—CH₂), 1.41 (t, 3H, CH₂—CH₃, J=8 Hz), 1.2 (s, 9H, (CH₃)₃); ¹³C NMR (CDCl₃, 100 MHz): 210.02 (CS), 196.67 (CO), 176.77 (O—CO), 139.75 (C—CO), 134.82 (C—Cl), 129.47 (2C, CH arom), 129.03 (2C, CH arom), 80.21 (CH—S), 70.33 (O—CH₂), 38.9 (C—(CH₃)₃), 34.18 (CO—CH₂), 28.46 (CH—CH₂), 27.01 (3C, (CH₃)₃), 13.73 (CH₂—CH₃); MS (ICP; m/z): 403 and 405 (M⁺), 301 and 303 (MH⁺—OPiv); IR (cm⁻¹, CCl₄): 1738 (O—C═O), 1692 (C═O), 1229 (C═S), 1050 (S—C).

### Example 5

### 1-Ethoxythiocarbonylsulfanyl-4-(4-fluorophenyl)-4-oxobutyl 2,2-dimethylpropionate

According to general method 2, a solution of 5 g (19.3 mmol) of xanthate of example 2 and of 5.72 ml (4.9 g, 38.7 mmol) of vinyl pivalate in 19 ml of 1,2-dichloroethane is brought to reflux and treated with DLP. The title product is obtained after silica gel chromatography (eluent: petroleum ether/ethyl acetate (95:5)) with a 90% yield (yellow oil). ¹H NMR (CDCl₃, 400 MHz): 7.97 (dd, 2H, CH arom, J=8.8 and 5.2 Hz), 7.14 (t, 2H, CH arom, J=8.2 Hz), 6.71 (t, 1H, CH—S, J=8 Hz), 4.63 (m, 2H, O—CH₂), 3.1 (dt, 2H, CO—CH₂, J=7.5 and 2.9 Hz), 2.40 (m, 2H, CH—CH₂), 1.42 (t, 3H, CH₂—CH₃, J=8 Hz), 1.2 (s, 9H, (CH₃)₃); ¹³C NMR (CDCl₃, 100 MHz): 210.16 (CS), 196.4 (CO), 176.88 (O—CO), 167.2; 164.6 (d, 1C, C—F, ¹J_{C-F} =255 Hz), 116.22 (C—CO), 130.81 (CH arom), 130.72 (CH arom), 116.0 (CH arom), 115.79 (CH arom), 80.36 (CH—S), 70.40 (O—CH₂), 45.53 (C—(CH₃)₃), 34.19 (CO—CH₂), 28.61 (CH—CH₂), 27.07 (3C, (CH₃)₃), 13.79 (CH₂—CH₃).

### Example 6

### 1-Ethoxyhiocarbonylsulfanyl-4-(4-methoxyphenyl)-4-oxobutyl2,2-dimethylpropionate

According to general method 2, 0.5 g (1.85 mmol) of xanthate of example 3 and 0.55 ml (2.7 mmol) of vinyl pivalate are dissolved in 2 ml of 1,2-dichloroethane. The product is purified by silica gel chromatography (eluent: petroleum ether/ethyl acetate (95:5)) so as to give the title product with an 86% yield (yellow oil). ¹H NMR (CDCl₃, 400 MHz): 7.92 (d, 2H, CH arom, J=8.8 Hz), 6.93 (d, 2H, CH arom, J=8.8 Hz), 6.71 (t, 1H, CH—S, J=8 Hz), 4.65-4.59 (m, 2H, O—CH₂), 3.87 (s, 3H, OCH₃), 3.08 (dq, 2H, CO—CH₂, J=7.2 and 4.4 Hz), 2.42-2.35 (m, 2H, CH—CH₂), 1.41 (t, 3H, CH₂—CH₃, J=6 Hz), 1.19 (s, 9H, (CH₃)₃); ¹³C NMR (CDCl₃, 100 MHz): 210.3 (CS), 196.6 (CO), 176.9 (O—CO), 163.7 (C—CO), 130.9 (C—OMe), 130.4 (CH arom), 113.9 (CH arom), 80.4 (CH—S), 70.4 (O—CH₂), 55.6 (OCH₃), 43.5 (C—(CH₃)₃), 33.9 (CO—CH₂), 28.8 (CH—CH₂), 27.1 (3C, (CH₃)₃), 13.8 (CH₂—CH₃); MS (ICP; m/z): 416 (MH⁺+NH₃), 399 (MH⁺), 297 (MH⁺−Opiv); IR (cm⁻¹, CCl₄): 1738 (O—C═O), 1683 (C═O), 1229 (C═S), 1051 (S—(S)C—O).

### Example 7

### 5-[1-Cyanomethyl-4-(4-fluorophenyl)-4-oxobutyl]-O-ethyl dithio-carbonate

According to general method 2, a solution of 2 g (7.74 mmol) of xanthate of example 2 and of 1.25 ml (1.03 g, 15.48 mmol) of allyl cyanide in 8 ml of 1,2-dichloroethane is brought to reflux and treated with DLP. The title product is obtained after silica gel chromatography (eluent: petroleum ether/ethyl acetate (9:1)) with an 81% yield (yellow oil). ¹H NMR (CDCl₃, 400 MHz): 7.98 (dd, 2H, CH arom, J=8.4 and 5.2 Hz), 7.14 (t, 2H, CH arom, J=8.4 Hz), 4.63 (ddd, 2H, O—CH₂, J=10.2 and 7.1 and 1.4 Hz), 4.01 (dddd, 1H, CH—S, J=15.2, 5.3, 5.3 and 5.3 Hz), 3.19 (t, 2H, CO—CH₂, J=7.2 Hz), 2.96 (t, 2H, CH₂—CN), 2.39 (ddt, 1H, CO—CH₂—CH₂, J=10.9, 7.2 and 4.3 Hz), 2.15 (dddd, 1H, CO—CH₂—CH₂, J=18, 7.2, 6.8 and 6.8 Hz), 1.42 (t, 3H, CH₂—CH₃, J=7 Hz); ¹³C NMR (CDCl₃, 100 MHz): 212.03 (CS), 196.5 (CO), 165.97 (d, 1C, C—F, ¹J_{C-F}=255.2 Hz), 132.93 (C—CO), 130.7 (d, 2C, CH arom, ²J_{C-F}=13 Hz), 117.08 (C≡N), 115.9 (d, 2C, CH arom, ³J_{C-F}=22 Hz), 70.74 (O—CH₂), 46.24 (CH—S), 35.42 (CO—CH₂), 26.72 (CH₂—CN), 24.5 (CO—CH₂—CH₂), 13.81 (CH₂—CH₃); MS (ICP; m/z): 342 (MH⁺+NH₃), 325 (MH⁺), 205 (MH⁺−SC(S)OEt); IR (cm⁻¹, CCl₄): 2250 (C≡N), 1741 (C═O), 1236 (C═S), 1051 (S—(S)C—O).

Preparation of the Tetralones of Formula (IVA) or (WB)

General Method 3:

A solution of 1 mmol of compound of formula (HIA) and of 0.1 mmol of camphorsulfonic acid (CSA) in 1,2-dichloroethane (10 ml) is brought to reflux and degassed under an argon atmosphere. After 15 minutes at reflux, 0.2 mmol of DLP is added to the reaction mixture and 0.2 mmol every hour until the starting product has been completely used up. When the reaction is complete, the solvent is evaporated off under vacuum and the product is purified by chromatography.

### Example 8

### 7-Chloro-4-oxo-1,2,3,4-tetrahydronaphthalen-1-yl 2,2-dimethyl-propionate

According to general method 3, a solution of 3.5 g (8.67 mmol) of compound of example 4 and of 0.2 g (0.86 mmol) of CSA in 87 ml of 1,2-dichloroethane is brought to reflux and treated with DLP. The title product is purified on a silica gel column, eluent: petroleum ether/ethyl acetate (9:1), and recrystallized with petroleum ether so as to obtain a slightly yellow solid (m.p.=76-80° C.) with an 84% yield. ¹H NMR (CDCl₃, 400 MHz): 8.0 (d, 1H, CH arom, J=8 Hz), 7.42 (d, 1H, CH arom, J=8 Hz), 7.41 (s, 1H, CH arom), 6.05 (dd, 1H, CH—Opiv, J=8 and 4 Hz), 2.9 (ddd, 1H, CO—CH₂, J=18, 10 and 4 Hz), 2.69 (ddd, 1H, CO—CH₂, J=20, 8 and 4 Hz), 2.41 (m, 1H, CO—CH₂—CH₂), 2.26 (m, 1H, CO—CH₂—CH₂), 1.25 (s, 9H, CH₃); ¹³C NMR (CDCl₃, 100 MHZ) 195.32 (CO), 177.34 (O—CO), 142.5 (C—CO), 139.94 (C—Cl), 129.92 (C—C—CO), 128.91 (CH arom), 128.64 (CH arom), 127.45 (CH arom), 67.99 (CH—Opiv), 38.66 (C—(CH₃)₃), 34.24 (CO—CH₂), 28.14 (CO—CH₂—CH₂), 27.14 (3C, CH₃); MS (ICP; m/z): 297 and 299 (MH⁺+NH₃), 281 and 283 (MH⁺), 180 and 182 (MH⁺−Opiv); IR (cm⁻¹, CCl₄): 1733 (O—C═O), 1696 (C═O), 1143 (O—C═O); microanalysis calculated for C₁₅H₁₇O₃Cl: C, 64.17; H, 6.103. Found: C, 64.04; H, 6.25.

### Example 9

### 7-Fluoro-4-oxo-1,2,3,4-tetrahydronaphthalen-1-yl 2,2-dimethyl-propionate

According to general method 3, a solution of 3 g (7.76 mmol) of compound of example 5 and of 0.18 g (0.77 mmol) of CSA in 78 ml of 1,2-dichloroethane is brought to reflux and treated with DLP. The title product (yellow oil) is obtained with a 54% yield after purification by silica gel chromatography, eluent: petroleum ether/ethyl acetate (9:1). ¹H NMR (CDCl₃, 400 MHz): 7.97 (dd, 1H, CH arom, J=8.8 and 5.2 Hz), 7.12 (m, 2H, CH arom), 6.06 (dd, 1H, CH—Opiv, J=8 and 4 Hz), 2.89 (ddd, 1H, CO—CH₂, J=20, 8 and 4 Hz), 2.68 (ddd, 1H, CO—CH₂, J=16.8, 8 and 4 Hz), 2.42 (m, 1H, CO—CH₂—CH₂), 2.24 (m, 1H, CO—CH₂—CH₂), 1.25 (s, 9H, (CH₃)₃); ¹³C NMR (CDCl₃, 100.5 MHz): 195.4 (CO), 176.3 (O—CO), 144.5 (C—F), 130.59 (d, 1C, CH arom, ³J_{C-F}=10.5 Hz), 116.41 (d, 1C, CH arom, ²J_{C-F}=23 Hz), 115.94 (C—CO), 115.50 (C—CCO), 114.4 (d, 1C, CH arom, ²J_{C-F}=23 Hz), 68.62 (CH—Opiv), 39.09 (C—(CH₃)₃), 34.75 (CO—CH₂), 28.7 (CO—CH₂—CH₂), 27.17 (3C, (CH₃)₃).

### Example 10

### 7-Methoxy-4-oxo-1,2,3,4-tetrahydronaphthalen-1-yl2,2-dimethyl-propionate

According to general method 3, 3 g (7.5 mmol) of compound of example 6 are dissolved in 75 ml of 1,2-dichloroethane and treated with DLP. The title product is purified on a silica gel column, eluent: petroleum ether/ethyl acetate (9:1), and recrystallized with ethanol so as to obtain a yellow solid (m.p.=80° C.) with a 30% yield. ¹H NMR (CDCl₃, 400 MHz): 8.06 (d, 1H, CH arom, J=8 Hz), 6.96 (dd, 1H, CH arom, J=8 and 4 Hz), 6.89 (d, 1H, CH arom, J=4 Hz), 6.08 (dd, 1H, CH—Opiv, J=8 and 4 Hz), 3.89 (s, 3H, OCH₃), 2.87 (ddd, 1H, CO—CH₂, J=18, 9 and 6 Hz), 2.66 (ddd, 1H, CO—CH₂, J=16, 8 and 4 Hz), 2.45-2.37 (m, 1H, CO—CH₂—CH₂), 2.29-2.21 (m, 1H, CO—CH₂—CH₂), 1.26 (s, 9H, CH₃); ¹³C NMR (CDCl₃, 100.5 MHz): 195.75 (CO), 177.91 (O—CO), 163.99 (C—CO), 143.7 (C—OMe), 129.85 (CH arom), 125.47 (C—C—CO), 114.94 (CH arom), 111.92 (CH arom), 69.13 (CH—Opiv), 55.59 (OCH₃), 39.07 (C—(CH₃)₃), 34.61 (CO—CH₂), 28.77 (CO—CH₂—CH₂), 27.18 (3C, CH₃); MS (ICP; m/z): 294 (MH⁺+NH₃), 277 (MH⁺), 176 (MH⁺+−OPiv); IR (cm⁻¹, CCl₄): 1731 (O—C═O), 1687 (C═O), 1146 (O—C═O); microanalysis calculated for C₁₆H₂₀O₄: C, 69.54; H, 7.3. Found: C, 69.07; H, 7.27.

### Example 11

### 7-Fluoro-4-oxo-1,2,3,4-tetrahydronaphthalen-1-yl)acetonitrile

According to general method 3, a solution of 2 g (6.14 mmol) of compound of example 7 and of 0.143 g (0.61 mmol) of CSA in 61 ml of 1,2-dichloroethane is brought to reflux and treated with DLP. The title product is purified on a silica gel column, eluent: petroleum ether/ethyl acetate (8:2), and recrystallized with petroleum ether so as to obtain a yellow solid (m.p.=126-128° C.) with a 36% yield. ¹H NMR (CDCl₃, 400 MHz): 8.12 (dd, 1H, CH arom, J=9 and 5.8 Hz), 7.1 (dt, 1H, CH arom, J=8.2 and 2.4 Hz), 7.05 (d, 1H, CH arom, J=9.6 Hz), 3.38 (tt, 1H, CH, J=11.6 and 6.1 Hz), 2.75-2.83 (m, 3H, CO—CH₂ and CH₂—CN), 2.68 (ddd, 1H, CH₂—CN, J=18, 7.4 and 5 Hz), 2.45 (dddd, 1H, CO—CH₂—CH₂, J=19, 9.4 and 4.6 Hz), 2.2-2.28 (m, 1H, CO—CH₂—CH₂); ¹³C NMR (CDCl₃, 100.5 MHz): 194.9 (CO), 166.06 (d, 1C, C—F, ¹J_{C-F}=256 Hz), 146.05 (C—CO), 131.3 (d, 1C, CH arom, ³J_{C-F}=10.9 Hz), 121.98 (C-N), 117.64 (C—CCO), 115.86 (d, 1C, CH arom, ²J_{C-F}=23 Hz), 114.24 (d, 1C, CH arom, ²J_{C-F}=22 Hz), 35.14 (CH), 34.95 (CO—CH₂), 27.58 (CH₂—CN), 22.95 (CO—CH₂—CH₂); MS (ICP; m/z): 221 (MH⁺+NH₃), 204 (MH⁺); IR (cm⁻¹, CCl₄): 2254 (C≡N), 1693 (C═O), 1250(C—F); microanalysis calculated for C₁₂H₁₀NOF: C, 70.93; H, 4.96; N, 6.44. Found: C, 70.53; H, 5.03; N, 6.56.

Preparation of the Oximes of Formula (VA) or (VB)

General Method 4:

Added to a solution containing 1 mmol of tetralone of formula (IVA) or (IVB) in ethanol (0.75 ml) is another solution made up of 1.3 equivalents of NH₂OH.HCl and 1.2 equivalents of sodium acetate in water (0.3 ml). The resulting solution is brought to reflux for 2 hours, then the ethanol is evaporated off under vacuum and the reaction mixture is extracted with ethyl acetate. The organic phase is dried over sodium sulfate, filtered, and concentrated under vacuum.

### Example 12

### 4-[(E)-Hydroxyjmino]-7-chloro-1,2,3,4-tetrahydronaphthalen-1-yl 2,2-dimethylpropionate

According to general method 4, a solution of 0.596 g (9.24 mmol) of NH₂OH.HCl and 1.16 g (8.53 mmol) of sodium acetate in 2.1 ml of water is added to a solution of 1.99 g (7.11 mmol) of tetralone of example 8 in 5.3 ml of ethanol. The mixture is brought to reflux and treated in the manner described. The title product is then recrystallized from petroleum ether and a yellow solid (m.p.=110-112° C.) is isolated with a 96% yield. ¹H NMR (CDCl₃, 400 MHz): 9.19 (broad s, 1H, OH), 7.86 (d, 1H, CH arom, J=8 Hz), 7.35 (d, 1H, CH arom, J=4 Hz), 7.29 (dd, 1H, CH arom, J=8 and 4 Hz), 5.89 (t, 1H, CH—Opiv, J=4 Hz), 2.93 (t, 2H, C(NOH)—CH₂, J=6 Hz), 2.07 (m, 2H, CH—CH₂), 1.22 (s, 9H, (CH₃)₃); ¹³C NMR (CDCl₃, 100 MHz): 178.03 (O—CO), 153.35 (C—NOH), 138.27 (C—C(NOH)), 135.65 (C—Cl), 129.04 (CH arom), 128.87 (C—C—C(NOH)), 128.0 (CH arom), 125.69 (CH arom), 68.99 (CH—Opiv), 39.09 (C—(CH₃)₃), 27.19 (3C, (CH₃)₃), 26.33 (C(NOH)—CH₂), 19.33 (CH—CH₂).

### Example 13

### 4-[(E)-Hydroxyimino]-7-fluoro-1,2,3,4-tetrahydronaphthalen-1-yl 2,2-dimethylpropionate

According to general method 4, a solution of 0.362 g (5.6 mmol) of NH₂OH.HCl and 0.425 g (5.17 mmol) of sodium acetate in 1.3 ml of water is added to a solution of 1.14 g (4.31 mmol) of tetralone of example 9 in 3.2 ml of ethanol. The reaction is brought to reflux and treated in the manner described. The product is then recrystallized from petroleum ether and a yellow solid (m.p.=125-128° C.) is isolated with a 92% yield. ¹H NMR (CDCl₃, 400 MHz): 9.10 (broad s, 1H, OH), 7.92 (dd, 1H, CH arom, J=8.8 and 6 Hz), 7.05 (dt, 1H, CH arom, J=8.5 and 2.5 Hz), 7.02 (dd, 1H, CH arom, J=8.2 and 3 Hz), 5.89 (dd, 1H, CH—OPiv, J=7 and 3.8 Hz), 2.94 (m, 2H, C(NOH)—CH₂), 2.07 (m, 2H, CH—CH₂), 1.23 (s, 9H, CH₃); ¹³C NMR (CDCl₃, 100.5 MHz): 178.04 (O—CO), 164.7; 162.21 (d, 1C, C—F, ¹J_{C-F}=250 Hz), 153.32 (C(NOH)), 139.2 (d, 1C, CH arom, ³J_{C-F}=8 Hz), 126.55 (C—C(NOH)), 116.19 (d, 1C, CH arom, ²J_{C-F}=22 Hz), 114.35 (d, 1C, CH arom, ²J_{C-F}=23 Hz), 69.13 (CH—Opiv), 39.08 (C—(CH₃)₃), 27.18 (3C, (CH₃)₃), 26.47 (C(NOH)—CH₂), 19.48 (CH—CH₂); MS (ICP; m/z): 297 (MH⁺+NH₃), 280 (MH⁺), 179 (MH⁺−Opiv); IR (cm⁻¹, CCl₄): 3593 (N—OH), 3300 (OH, hydrogen bond), 1730 (O—C═O), 1279 (O—C═O).

### Example 14

### 4-[(E)-Hydroxyimio]-7-methoxy-1,2,3,4-tetrahydronaphthalen-1-yl 2,2-dimethylprop)ionate

According to general method 4, 0.065 g (1.011 mmol) of hydroxylamine hydrochloride and 0.127 g (0.933 mmol) of sodium acetate are dissolved in 0.2 ml of water. This solution is added to a solution containing 0.215 g (0.778 mmol) of tetralone of example 10 in 0.6 ml of ethanol and brought to reflux. After treatment, the title product is recrystallized with petroleum ether and a yellow solid (m.p.=114-115° C.) is obtained with a 92% yield. ¹H NMR (CDCl₃, 400 MHz): 9.34 (broad s, 1H, OH), 7.86 (d, 1H, CH arom, J=8 Hz), 6.9 (d, 1H, CH arom, J=2 Hz), 6.87 (dd, 1H, CH arom, J=7.8 and 2.2 Hz), 5.91 (dd, 1H, CH—Opiv, J=6 and 4 Hz), 3.82 (s, 3H, OCH₃), 2.93 (t, 2H, C(NOH)—CH₂, J=6.8 Hz), 2.04-2.11 (m, 2H, CH—CH₂), 1.22 (s, 9H, (CH₃)₃); ¹³C NMR (CDCl₃, 100.5 MHz): 178.13 (O—CO), 160.66 (C—NOH), 153.75 (C—C(NOH)), 138.38 (C—OMe), 125.8 (CH arom), 122.99 (C—C—C(NOH)), 115.38 (CH arom), 112.15 (CH arom), 69.65 (CH—Opiv), 55.44 (OCH₃), 39.09 (C—(CH₃)₃), 27.22 (3C, (CH₃)₃), 26.63 (C(NOH)—CH₂), 19.43 (CH—CH₂); MS (ICP; m/z): 292 (MH⁺), 191 (MH⁺−Opiv); IR (cm⁻¹, CCl₄): 3596 (N—OR), 1727 (O—C═O), 1604 (C═N—OH), 1272 (O—C═O).

### Example 15

### {7-Fluoro-4-[(E)-hvdroxyimino]-1,2,3,4-tetrahydronaphthalen-1-yl}-acetonitrile

According to general method 4, a solution of 0.165 g (2.55 mmol) of NH₂OH.HCl and 0.321 g (2.36 mmol) of sodium acetate in 0.4 ml of water is added to a solution of 0.4 g (1.96 mmol) of tetralone of example 11 in 0.9 ml of ethanol. The reaction is brought to reflux and treated in the manner described. The title product is then recrystallized from ethyl acetate/petroleum ether so as to give a yellow solid (m.p.=167-168° C.) with a 78% yield. ¹H NMR (CDCl₃, 400 MHz): 9.94 (broad s, 1H, OH), 7.97 (dd, 1H, CH arom, J=8.8 and 6 Hz), 6.93-7.0 (m, 2H, CH arom), 3.16-3.22 (m, 1H, CH), 2.96 (dt, 1H, C(NOH)—CH₂, J=18.8 and 5.2 Hz), 2.73 (dtd, 1H, C(NOH)—CH₂, J=27, 8.3 and 2 Hz), 2.58-2.66 (m, 1H, CH₂—CN), 2.05-2.12 (m, 2H, CH—CH₂); ¹³C NMR (CDCl₃, 100 MHz): 162.3 (d, 1C, C—F, ¹J_{C-F}=250 Hz), 150.71 (C═NOH), 140.02 (C—C═NOH), 126.8 (C≡N), 126.24 (d, 1C, CH arom, ³J_{C-F}=7.7 Hz), 117.77 (C—CH), 114.36 (d, 1C, CH arom, ²J_{C-F}=15 Hz), 113.56 (d, 1C, CH arom, ²J_{C-F}=23 Hz), 34.73 (CH), 24.8 (C(NOH)—CH₂), 21.93 (CH₂—CN), 19.15 (CH—CH₂); MS (ICP; m/z): 236 (MH⁺+NH₃), 219 (MH⁺); IR (cm⁻¹, CCl₄): 3591 (N—OH), 1741 (C═N—OH), 1239 (O—C═O).

Preparation of the Benzazepines of Formula (IA) or (IB)

General Method 5:

A solution of 1 mmol of the oxime of formula (VA) or (VB) in 10 ml of dichloromethane is added dropwise to another solution containing 4 mmol of PCl₅ in dichloromethane (10 ml) at 0° C. The solution is then stirred at ambient temperature for 2 hours and is then neutralized with an aqueous solution of NaHCO₃, extracted with CH₂Cl₂, dried over sodium sulfate, filtered, and concentrated. The oil thus obtained is solubilized in 10 ml of acetic acid and brought to reflux, and then 6 mmol of powdered Zn are added slowly to this solution and the reflux is maintained for 30 minutes. The resulting mixture is then diluted with ethyl acetate, filtered over celite, washed with a saturated solution of NaHCO₃ and concentrated under vacuum. The product obtained in this manner is then solubilized in 1.5 ml of THF and added dropwise to a solution of 2 mmol of BH₃.THF complex in 1.5 ml of THF at 0° C. The solution is brought to reflux for 30 minutes and left to cool, it is then treated with a few drops of a saturated solution of acetic acid, the THF is evaporated off, and the aqueous phase is basified with an aqueous solution of Na₂CO₃ and extracted with CH₂Cl₂. The residue obtained is purified by chromatography.

### Example 16

### 7-Chloro-2,3,4,5-tetrahydro-1H-benzo[b]azepin-5-yl 2,2-dimethyl-propionate ester

According to general method 5, a solution of 1.1 g (3.71 mmol) of oxime of example 12 in 37 ml of CH₂Cl₂ is treated with a solution of 3.1 g (14.8 mmol) of PCl₅ in 37 ml of CH₂Cl₂. The residue thus isolated is solubilized in 35 ml of acetic acid and then 1.4 g (21.45 mmol) of Zn are added. A solution of 1.1 g (3.71 mmol) of the product thus obtained in 5.5 ml of THF is reduced with a 1 M solution of BH₃-THF in 5.5 ml of THF according to the general process. The title product is purified on a silica gel column, eluent: petroleum ether/ethyl acetate (9:1), so as to obtain a white solid (m.p.=65-66° C.) with a 40% yield over the 3 stages. ¹H NMR (CDCl₃, 400 MHz): 7.27 (d, 1H, CH arom, J=2.4 Hz), 7.02 (dd, 1H, CH arom, J=8.4 and 2.4 Hz), 6.64 (d, 1H, CH arom, J=8.8 Hz), 5.84 (d, 1H, CH—Opiv, J=8 Hz), 3.83 (broad s, 1H, NH), 3.25 (dt, 1H, NH—CH₂, J=13.2 and 4.2 Hz), 2.93 (ddd, 1H, NH—CH₂, J=13.2, 9.6 and 3.2 Hz), 1.8-2.0 (m, 4H, NH—CH₂ and NH—CH₂—CH₂), 1.27 (s, 9H, (CH₃)₃); ¹³C NMR (CDCl₃, 100 MHz): 177.35 (O—CO), 147.5 (C—Cl), 131.54 (C—NH), 128.14 (CH arom), 127.74 (CH arom), 125.23 (C—CNH), 120.75 (CH arom), 73.72 (CH—Opiv), 47.53 (NH—CH₂), 41.7 (C—(CH₃)₃), 31.3 (CH—CH₂), 27.31 (3C, (CH₃)₃), 27.0 (NH—CH₂—CH₂); MS (ICP; m/z): 282 and 284 (MH⁺), 181 and 184 (MH⁺−Opiv); IR (cm⁻¹, CCl₄): 3386 (NH), 1729 (O—C═O), 1151 (O—C═O); microanalysis calculated for C₁₅H₂₀NO₂Cl: C, 63.94; H, 7.15. Found: C, 63.75; H, 7.15.

### Example 17

### 7-Fluoro-2,3,4,5-tetrahydro-1H-benzo[b]azeyin-5-yl 2,2-dimethyl-propionate

According to general method 5, a solution of 0.956 g (3.42 mmol) of oxime of example 13 in 34 ml of CH₂Cl₂ is treated with a solution of 2.85 g (13.6 mmol) of PCl₅ in 34 ml of CH₂Cl₂. The residue thus obtained is solubilized in 34 ml of acetic acid and 1.34 g (20.5 mmol) of Zn are added. A solution of 0.875 g (3.13 mmol) of product thus obtained in 4.7 ml of THF is reduced with a 1 M solution of BH₃-THF in 4.7 ml of THF according to the general process. The title product is purified on a silica gel column, eluent: petroleum ether/ethyl acetate (9:1), so as to obtain a white solid (m.p.=58-60° C.) with a 61% yield over the 3 stages. ¹H NMR (CDCl₃, 400 MHz): 7.05 (dd, 1H, CH arom, J=9.4 and 3 Hz), 6.78 (td, 1H, CH arom, J=8.3 and 3.1 Hz), 6.68 (dd, 1H, CH arom, J=8.4 and 4.8 Hz), 5.84 (d, 1H, CH—Opiv, J=10 Hz), 3.58 (broad s, 1H, NH), 3.26 (dt, 1H, NH—CH₂, J=13.2 and 4.2 Hz), 2.79 (td, 1H, NH—CH₂, J=11.7 and 2.8 Hz), 1.7-2.0 (m, 4H, CH—CH₂ and NH—CH₂—CH₂), 1.26 (s, 9H, (CH₃)₃); ¹³C NMR (CDCl₃, 100.5 MHz): 177.2 (O—CO), 157.7 (d, 1C, C—F, ¹J_{C-F}=236 Hz), 144.4 (C—NH), 133.19 (C—CH), 120.94 (d, 1C, CH arom, ²J_{C-F}=7 Hz), 114.06 (d, 1C, CH arom, ²J_{C-F}=7 Hz), 113.71 (d, 1C, CH arom, ³J_{C-F}=24 Hz), 73.57 (CH—Opiv), 47.57 (NH—CH₂), 39.04 (C—(CH₃)₃), 32.03 (NH—CH₂—CH₂), 27.52 (CH—CH₂), 27.31 (3C, (CH₃)₃); MS (ICP; m/z): 266 (MH⁺), 165 (MH⁺−Opiv); IR (cm⁻¹, CCl₄): 3385 (NH), 1729 (O—C═O), 1152 (O—C═O); microanalysis calculated for C₁₅H₂₀NO₂F: C, 67.9; H, 7.6. Found: C, 67.93; H, 7.62.

### Example 18

### 7-Methoxy-2,3,4,5-tetrahydro-1H-benzo[b]azepin-5-yl 2,2-dimethyl-propionate

According to general method 5, a solution of 0.1 g (0.343 mmol) of oxime of example 14 in 3.4 ml of CH₂Cl₂ is treated, firstly, with 0.286 g (1.37 mmol) of PCl₅ in dichloromethane (3.4 ml). Secondly, the reaction crude thus obtained is solubilized in acetic acid (3.4 ml) and 0.135 g (2.05 mmol) of powdered zinc is added. Finally, the amide is reduced with 0.68 ml (0.68 mol) of 1 M solution of BH₃-THF in 1 ml of THF. The title product is purified on a silica gel column, eluent: petroleum ether/ethyl acetate (9:1), so as to obtain a yellow oil with a 21% yield over the 3 stages. ¹H NMR (CDCl₃, 400 MHz): 6.93 (d, 1H, CH arom, J=2.4 Hz), 6.66-6.7 (m, 2H, CH arom), 5.87 (d, 1H, CH—Opiv, J=9.2 Hz), 3.76 (s, 3H, OCH₃), 3.24 (dt, 1H, NH—CH₂, J=12.8 and 4.2 Hz), 2.78 (td, 1H, NH—CH₂, J=11.7 and 2.8 Hz), 1.75-2.0 (m, 4H, CH—CH₂ and NH—CH₂—CH₂), 1.28 (s, 9H, (CH₃)₃); ¹³C NMR (CDCl₃, 100 MHz): 189.11 (O—CO), 141.93 (C—OMe), 134.04 (C—NH), 130.24 (C—CH), 120.99 (CH arom), 112.97 (CH arom), 112.49 (CH arom), 74.06 (CH—Opiv), 55.57 (OCH₃), 48.23 (NH—CH₂), 39.1 (C—(CH₃)₃), 32.33 (NH—CH₂—CH₂), 27.73 (CH—CH₂), 27.38 (3C, (CH₃)₃); MS (ICP; m/z): 278 (MH⁺), 177 (MH⁺−Opiv); IR (cm⁻¹, CCl₄): 3450 (NH), 1727 (O—C═O), 1156 (O—C═O).

### Example 19

### (7-Fluoro-2,3,4,5-tetrahydro-1H-benzo[b]azepin-5-yl)acetonitrile

According to general method 5, a solution of 0.25 g (1.14 mmol) of oxime of example 15 in 12 ml of CH₂Cl₂ is treated with a solution of 0.954 g (4.58 mmol) of PCl₅ in 12 ml of CH₂Cl₂. The residue thus obtained is solubilized in 12 ml of acetic acid and 0.45 g (6.87 mmol) of Zn are added. A solution of 0.25 g (1. 14 mmol) of product thus obtained in 2 ml of THF is reduced with 2.3 ml (2.3 mmol) of 1 M solution of BH₃.THF in 2 ml of THF according to the general process. The title product is purified on a silica gel column, eluent: petroleum ether/ethyl acetate (9:1), so as to obtain a white solid (m.p.=74-75° C.) with a 39% yield over the 3 stages. ¹H NMR (CDCl₃, 400 MHz): 6.84 (dd, 1H, CH arom, J=9.2 and 2.4 Hz), 6.78 (td, 1H, CH arom, J=8.2 and 3 Hz), 6.67 (dd, 1H, CH arom, J=8.4 and 4.8), 3.59 (broad s, 1H, NH), 3.19-3.29 (m, 2H, CH and NH—CH₂), 3.0 (dd, 1H, CH₂—CN, J=16.6 and 8.6 Hz), 2.81 (dd, 1H, CH₂—CN, J=16.6 and 7.4 Hz), 2.72 (ddd, 1H, NH—CH₂, J=12.6, 10.8 and 2 Hz), 2.05-2.1 (m, 1H, NH—CH₂—CH₂), 1.84-1.95 (m, 1H, NH—CH₂—CH₂), 1.71-1.79 (m, 2H, CH—CH₂); ¹³C NMR (CDCl₃, 100 MHz): 157.78 (d, 1C, C—F, ¹J_{C-F}=241 Hz), 145.67 (C—NH), 134.36 (d, 1C, CH arom, ³J_{C-F}=6 Hz), 121.75 (C≡N), 119.17 (C—CH), 116.66 (d, 1C, CH arom, ²J_{C-F}=22.1 Hz), 114.25 (d, 1C, CH arom, ²J_{C-F}=19.1 Hz), 48.97 (NH—CH2), 42.49 (CH), 29.94 (CH₂—CN), 26.11 (NH—CH₂—CH₂), 19.12 (CH—CH₂); MS (ICP; m/z): 206 (MH⁺); IR (cm⁻¹, CCl₄): 3384 (NH), 2246 (C≡N), 1253 (O—C═O); microanalysis calculated for C₁₂H₁₃N₂F: C, 70.57; H, 6.42. Found: C, 70.42; H, 6.55.

### Example 20

### 3,3,7-Trichloro-2,3,4,5-tetrahydro-1H-benzo[b]lazepin-5-yl 2,2-di-methylpropionate

Added dropwise to a solution of 0.28 g (1.35 mmol) of PCl₅ in 1.5 ml of CH₂Cl₂ at 0° C. is a solution of 0.1 g (0.338 mmol) of oxime of example 12 in the same solvent. The reaction is then stirred at ambient temperature until the starting product has disappeared. The reaction is then cooled to 0° C. and a suspension of 0.128 g (3.38 mmol) of NaBH₄ in 0.3 ml of ethanol is added gently to the reaction medium. The stirring is maintained at 0° C. until the reaction is complete. Finally, the reaction mixture is extracted with dichloromethane. The organic phase is dried over sodium sulfate, filtered, and concentrated under vacuum. The residue thus obtained is purified by silica gel chromatography, eluent: petroleum ether/ethyl acetate (95:5), so as to provide a white solid (m.p.=103-104° C.) with a 48% yield over the 2 stages. ¹H NMR (CDCl₃, 400 MHz): 7.21 (d, 1H, CH arom, J=2 Hz), 7.10 (d, 1H, CH arom, J=8.8 and 2.4 Hz), 6.72 (d, 1H, CH arom, J=8.4 Hz), 6.03 (t, 1H, CH—Opiv, J=5.6 Hz), 4.22 (broad s, 1H, NH), 3.75 (dd, 1H, CH—CH₂, J=14.8 and 6.8 Hz), 3.57 (d, 1H, CH—CH₂, J=14 Hz), 3.86 (d, 2H, NH—CH₂, J=4 Hz), 1.29 (s, 9H, (CH₃)₃); ¹³C NMR (CDCl₃, 100 MHz): 177.0 (O—CO), 145.21 (C—Cl), 142.32 (C—NH), 128.52 (CH arom), 128.23 (CH arom), 126.62 (C—CH), 120.98 (CH arom), 88.36 (CCl₂), 69.19 (CH—Opiv), 61.65 (CH—CH₂), 50.08 (NH—CH₂), 38.97 (C—(CH₃)₃), 27.27 (3C, (CH₃)₃); MS (ICP; m/z): 350 and 352 (MH⁺), 248 and 250 (MH⁺−Opiv); IR (cm⁻¹, CCl₄): 3446 (NH), 1735 (O—C═O), 1139 (O—C═O); microanalysis calculated for C₁₅H₁₈NO₂Cl₃: C, 51.38; H, 5.17. Found: C, 51.31; H, 5.16.

General Method 6:

A solution containing X mmol of amine and 4 X mmol of triethyl-amine, dissolved in dichloromethane (5 ml/mmol), is stirred at 0° C. in a bath of ice-cold water. 3 X mmol of acid chloride dissolved in 5 ml/mmol of dichloromethane are added dropwise to this solution. The reaction mixture is left at 0° C. for 15 minutes, and is then allowed to return to ambient temperature. When the starting product has been completely used up (about 1 hour), the reaction is basified by adding a few drops of saturated Na₂CO₃ solution and the product is extracted with ethyl acetate. The solution is dried, filtered, concentrated under vacuum and purified by silica gel chromatography.

### Example 21

### 2,2-Dimethylpropionic acid 7-chloro-1-(2-methyl-4-nitrobenzoyl)-2,3,4,5-tetrahydro-1H-benzo[b]azepin-5-yl ester

According to general method 6, a solution of 0.212 g (1.06 mmol) of 2-methyl-4-nitrobenzoyl chloride in 2 ml of dichloromethane is added to a solution of 0.1 g (0.35 mmol) of the compound of example 16 and of 2 ml (0.143 g, 1.41 mmol) of triethylamine in 0.1 ml of dichloromethane. The residue thus obtained is purified by silica gel chromatography, eluent: petroleum ether/ethyl acetate (9:1), so as to give a white solid (m.p.=58-60° C.) with a 98% yield. ¹H NMR (CDCl₃, 400 MHz): 7.92 (d,1H, CH arom, J=2 Hz), 7.77 (dd, 1H, CH arom, J=8.2 and 2.2 Hz), 7.15 (d, 1H, CH arom, J=8.4), 7.14 (s, 1H, CH arom), 6.85 (dd, 1H, CH arom, J=8 and 2.4 Hz), 6.51 (d, 1H, CH arom, J=8.4 Hz), 5.95 (dd, 1H, CH—Opiv, J=5.6 and 2.6 Hz), 4.74 (dt, 1H, N—CH₂, J=14 and 4.1 Hz), 2.81 (ddd, 1H, N—CH₂, J=12, 10.1 and 2.1 Hz), 2.49 (s, 3H, Ar—CH₃), 2.09-2.19 (m, 2H, N—CH₂—CH₂), 1.67-1.82 (m, 2H, CH—CH₂), 1.29 (s, 9H, CH₃); ¹³C NMR (CDCl₃, 100 MHz): 177.2 (O—CO), 167.9 (N—CO), 147.7 (C—NO₂), 141.8 (C—NH), 140.2 (C—CO), 137.64 (C—Cl), 137.4 (C—CH), 134.28 (C—CH₃), 128.88 (CH arom), 128.0 (CH arom), 127.59 (CH arom), 125.26 (CH arom), 124.65 (CH arom), 121.0 (CH arom), 71.83 (CH—Opiv), 46.6 (N—CH₂), 39.14 (C—(CH₃)₃), 32.02 (N—CH₂—CH₂), 27.33 (3C, (CH₃)₃), 25.4 (CH—CH₂), 20.23 (Ar—CH₃); MS (ICP; m/z): 463 and 465 (MH⁺+NH₃), 446 and 448 (MH⁺), 342 and 344 (MH⁺−Opiv); IR (cm⁻¹, CCl₄): 1735 (O—C═O), 1659 (N—C═O), 1529 (NO₂), 1139 (O—C═O).

### Example 22

### 7-Fluoro-1-(2-methyl-4-nitrobenzoyl)-2,3,4,5-tetrahydro-1H-benzo[b]azepin-5-yl 2,2-dimethylpropionate

According to general method 6, a solution of 0.05 g (0.18 mmol) of benzazepine of example 17 and of 0.1 ml (0.076 g, 0.75 mmol) of triethylamine in 1 ml of dichloromethane is treated with 0.112 g (0.565 mmol) of 2-methyl-4-nitrobenzoyl chloride. The residue thus obtained is purified by silica gel chromatography, eluent: petroleum ether/ethyl acetate (9:1), so as to give a yellow oil with a 97% yield. ¹H NMR (CDCl₃, 400 MHz): 7.98 (d,1H, CH arom, J=2.4 Hz), 7.82 (dd, 1H, CH arom, J=8.6 and 2.6 Hz), 7.22 (d, 1H, CH arom, J=8.8), 6.94 (d, 1H, CH arom, J=8.8 Hz), 6.63 (d, 1H, CH arom, J=5.2 Hz), 6.63 (s, 1H, CH arom), 6.03 (dd, 1H, CH—Opiv, J=11.4 and 3 Hz), 4.81 (dt, 1H, N—CH₂, J=13.6 and 4.2 Hz), 2.88 (td, 1H, N—CH₂, J=12.1 and 2 Hz), 2.56 (s, 3H, Ar—CH₃), 2.16-2.25 (m, 2H, N—CH₂—CH₂), 1.75-1.9 (m, 2H, CH—CH₂), 1.36 (s, 9H, CH₃); ¹³C NMR (CDCl₃, 100 MHz): 177.2 (O—CO), 168.04 (N—CO), 162.04 (d, 1C, C—F, ¹J_{C-F}=248 Hz), 147.7 (C—NO₂), 142.05 (C—N), 137.58 (C—CO), 134.85 (C—CH), 129.38 (d, 1C, CH arom, ²J_{C-F}=10.5 Hz), 127.54 (d, 1C, CH arom), 125.22 (d, 1C, CH arom, ²J_{C-F}=4.2 Hz), 120.9 (CH arom), 120.2 (CH arom), 114.84 (C—CH₃), 111.63 (d, 1C, CH arom, ³J_{C-F}=28 Hz), 71.99 (CH—Opiv), 46.63 (N—CH₂), 39.06 (C—(CH₃)₃), 32.15 (N—CH₂—CH₂), 27.33 (3C, (CH₃)₃), 25.48 (CH—CH₂), 19.89 (Ar—CH₃); MS (ICP; m/z): 446 (MH⁺+NH₃), 429 (MH⁺), 326 (MH⁺−Opiv); IR (cm⁻¹, CCl₄): 1735 (O—C═O), 1659 (N—C═O), 1529 (NO₂), 1346 (NO₂), 1139 (O—C═O).

### Example 23

### 1-4-Amino-2-methylbenzoyl)-7-chloro-2,3,4,5-tetrahydro-1H-benzo[b]azepin-5-yl 2,2-dimethylpropionate

0.32 g (1.68 mmol) of SnCl₂ is added to a solution of 0.15 g (0.337 mmol) of the compound of example 21 in 0.6 ml of ethanol and 0.2 ml of concentrated HCl at reflux. The reaction is heated at this temperature until the starting product has been completely used up (1 h 30). The reaction is then left to return to ambient temperature, and the solution is basified by the addition of a saturated solution of Na₂CO₃, extracted with ethyl acetate, dried over Na₂SO₄, filtered, and concentrated under vacuum. The residue thus obtained is purified by silica gel chromatography, eluent: petroleum ether/ethyl acetate (4:1), so as to provide white crystals (m.p.=185-186° C.) with an 82% yield. ¹H NMR (CDCl₃, 400 MHz): 7.22 (s, 1H, CH arom), 6.93 (d, 1H, CH arom, J=8 Hz), 6.73 (d, 1H, CH arom, J=8), 6.56 (d, 1H, CH arom, J=8 Hz), 6.4-6.46 (m, 2H, CH arom), 6.22 (d, 1H, CH arom, J=8 Hz), 6.02 (d, 1H, CH—Opiv, J=7.4 Hz), 4.84 (d, 1H, N—CH₂, J=8.4 Hz), 3.7 (broad s, 2H, NH), 3.78 (t, 1H, N—CH₂, J=12 Hz), 2.37 (s, 3H, Ar—CH₃), 2.05-2.21 (m, 2H, N—CH₂—CH₂), 1.68-1.77 (m, 2H, CH—CH₂), 1.35 (s, 9H, CH₃); ¹³C NMR (CDCl₃, 100.5 MHz): 177.07 (O—CO), 170.4 (N—CO), 147.14 (C—CO), 139.34 (C—NCO), 137.75 (C—Cl), 132.65 (C—NH₂), 129.58 (CH arom), 128.71 (CH arom), 127.75 (CH arom), 125.57 (C—CH), 124.22 (CH arom), 116.62 (CH arom), 114.32 (C—CH₃), 111.88 (CH arom), 72.1 (CH—Opiv), 46.18 (N—CH₂), 39.05 (C—(CH₃)₃), 32.05 (N—CH₂—CH₂), 27.32 (3C, (CH₃)₃), 25.56 (CH—CH₂), 20.1 (Ar—CH₃); MS (ICP; m/z): 433 and 435 (MH⁺+NH₃), 416 and 418 (MH⁺), 312 and 314 (MH⁺−Opiv); IR (cm⁻¹, CCl₄): 3400 (NH₂), 1734 (O—C═O), 1651 (N—C═O), 1142 (O—C═O).

### Example 24

### 1-(4-Amino-2-methylbenzoyl)-7-fluoro-2,3,4,5-tetrahydro-1H-benzo[b]azepin-5-yl 2,2-dimethylpropionate

0.124 g (0.654 mmol) of SnCl₂ is added to a solution of 0.054 g (0.130 mmol) of the compound of example 22 in 0.3 ml of ethanol and 0.1 ml of concentrated HCl at reflux. The reaction is heated at this temperature until the starting product has been completely used up (1 h 30). The reaction is then left to return to ambient temperature, and the solution is basified by the addition of a saturated solution of Na₂CO₃, extracted with ethyl acetate, dried over Na₂SO₄, filtered, and concentrated under vacuum. The residue thus obtained is purified by silica gel chromatography, eluent: petroleum ether/ethyl acetate (4:1), so as to provide a colorless oil with a 78% yield. ¹H NMR (CDCl₃, 400 MHz): 6.95 (d, 1H, CH arom, J=9.6 Hz), 6.73 (d, 1H, CH arom, J=8 Hz), 6.56-6.66 (m, 2H, CH arom), 6.39 (s, 1H, CH arom), 6.20 (d, 1H, CH arom, J=8.4 Hz), 6.03 (d, 1H, CH—Opiv, J=10.4 Hz), 4.84 (d, 1H, N—CH₂, J=13.6 Hz), 3.71 (broad s, 2H, NH₂), 2.77 (t, 1H, N—CH₂, J=12.2 Hz), 2.36 (s, 3H, Ar—CH₃), 2.13-2.15 (m, 2H, N—CH₂—CH₂), 1.69-1.84 (m, 2H, CH—CH₂), 1.34 (s, 9H, CH₃); ¹³C NMR (CDCl₃, 100.5 MHz): 177.1 (O—CO), 170.53 (N—CO), 161.26 (d, 1C, C—F, ¹J_{C-F}=247 Hz), 150.33 (C—CO), 147.0 (CH arom), 140.08 (C—NCO), 137.62 (C—CH), 129.93 (C—NH₂), 128.46 (CH arom), 116.5 (d, 1C, CH arom, ²J_{C-F}=12.6 Hz), 114.44 (d, 1C, CH arom, ²J_{C-F}=6.03 Hz), 112.17 (C—CH), 111.12 (C—CH₃), 110.74 (d, 1C, CH arom, ³J_{C-F}=26 Hz), 72.24 (CH—Opiv), 46.19 (N—CH₂), 39.04 (C—(CH₃)₃), 32.17 (N—CH₂—CH₂), 27.32 (3C, (CH₃)₃), 25.69 (CH—CH₂), 19.34 (Ar—CH₃); MS (ICP; m/z): 399 (MH⁺), 298 (MH⁺-OPiv); IR (cm⁻¹, CCl₄): 3487 and 3399 (NH₂), 1734 (O—C═O), 1649 (N—C═O), 1148 (O—C═O).

### Example 25

Tolvaptan

According to general method 6, a solution of 0.05 g (0.12 mmol) of the compound of example 23 and of 0.07 ml (0.048 g, 0.48 mmol) of triethylamine in 1 ml of dichloromethane is treated with 0.046 g (0.301 mmol) of 2-methylbenzoyl chloride. The acylation reaction crude is then dissolved in 1.5 ml of ethanol and 2 ml of a 2N solution of NaOH are added to the reaction medium. The resulting solution is heated at 50° C. for 2 hours. After having allowed the reaction to cool, a few milliliters of water are added and the product precipitates. The crystals are filter-dried, washed with cold water and recrystallized with methanol/ether so as to give Tolvaptan with an 85% yield over the 2 stages. The spectroscopic characteristics of this product correspond to those reported in the literature.

### Example 26

Fluorotolvaptan

According to general method 6, a solution of 0.04 g (0.10 mmol) of the compound of example 24 and of 0.05 ml (0.04 g, 0.40 mmol) of triethylamine in 1 ml of dichloromethane is treated with 0.046 g (0.301 mmol) of 2-methylbenzoyl chloride. The acylation reaction crude is then dissolved in 1 ml of ethanol and 1.5 ml of a 2N solution of NaOH are added to the reaction medium. The resulting solution is heated at 50° C. for 2 hours. After having allowed the reaction to cool, a few milliliters of water are added and the product precipitates. The crystals are filter-dried, washed with cold water and recrystallized with methanol/ethyl ether so as to give the title product (white solid) with a quantitative yield over the 2 stages.

1-51. (canceled)

52. A method of preparing at least one benzazepine compound of general formula (IA): 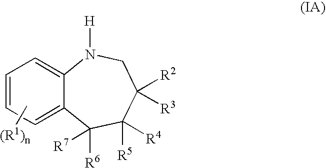
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR^{l}), —(CH₂)ₘ—SR^{k}, —(CH₂)ₘ—S(O)R^{k}, —(CH₂)ₘ—SO₂R^{k}, —(CH₂)ₘ—SO₂NR^{k}R^{l}, —(CH₂)ₘSO₃R^{k}, —(CH₂)ₘ—NO₂, —(CH₂)ₘ—CN, —(CH₂)ₘ—PO(OR^{k})(OR^{l}), —(CH₂)ₘ—SiR^{k}R^{l}R^{m}, —(CH₂)ₘ—COOR^{k}, —(CH₂)ₘ—NCOR^{k}, and —(CH₂)ₘ—NR^{k}R^{l}, and:
R^{k}, R^{l} and R^{m} are each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, and heterocycles,
or R^{k} and R^{l} form, together with the atom to which they are attached, a heterocycle,
with m denoting an integer greater than or equal to 0,
n represents an integer chosen from the group consisting of 0, 1, 2, 3 and 4, with, when n is greater than or equal to 2, r the corresponding R^{l} groups are identical or different, and optionally form, together, a hydrocarbon-based ring or a heterocycle,
R², R³, R⁴, R⁵, R⁶ and R⁷, independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituents groups chosen from the group consisting of —(CH₂)ₘ—OR^{k}, —CH(OR^{k})(OR^{l}), —(CH₂)ₘ—SR^{k}, —(CH₂)ₘ—S(O)R^{k}, —(CH₂)ₘ—SO₂R^{k}, —(CH₂)ₘ—SO₂NR^{k}R^{l}, —(CH₂)ₘ—SO₃R^{k}, —(CH₂)ₘ-NO₂, —(CH₂)ₘ—CN, —(CH₂)ₘ—PO(OR^{k})(OR^{l}), —(CH₂)ₘ—SiR^{k}R^{l}R^{m}, —(CH₂)ₘ—COOR^{k}, —(CH₂)ₘ—NCOR^{k}, and —(CH₂)ₘ—NR^{k}R^{l}, with R^{k}, R^{l}, R^{m} and m as defined above,
or R⁴, R⁵, R⁶ and R⁷ form, in pairs, one or more hydrocarbon-based ring(s) or heterocycle(s), with at least one of the R⁴, R⁵, R⁶ and R⁷ groups representing a hydrogen atom,
from at least one compound of general formula (IIA) 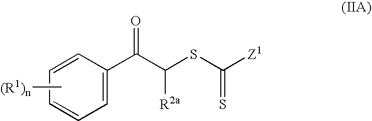
in which
Z¹ represents a group chosen from the group consisting of:
(i) alkyl groups, acyl groups, aryl groups, aralkyl groups, alkene groups, alkyne groups, hydrocarbon-based rings, and heterocycles,
(ii) —OR^{a} or —SR^{a} groups in which R^{a} is a group chosen from the group consisting of:
alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, and polymer chains;
—CR^{b}R^{c}PO(OR^{d})(OR^{e}) groups in which:
R^{b} and R^{c} are each, independently of one another, chosen from the group consisting of hydrogen atom, halogen atoms, alkyl groups, perfluoroalkyl groups, hydrocarbon-based rings, heterocycles, —NO₂ groups, —NCO groups, —CN groups, and groups chosen from the group consisting of —R^{f}, —SO₃R^{f}, —OR^{f}, —SR^{f}, —NR^{f}R^{g}, —COOR^{f}, —O₂CR^{f}, —CONR^{f}R^{g}, —NR^{f}COR^{g}, in which R^{f} and R^{g} are each independently chosen from the group consisting of alkyl groups, alkenyl groups, alkynyl groups, cycloalkenyl groups, cycloalkynyl groups, and aryl groups optionally condensed with a heterocycle, alkaryl, arylalkyl or heteroaryl,
or R^{b} and R^{c} form, together with the carbon atom to which they are attached, a C═O group, a C═S group, a hydrocarbon-based ring or a heterocycle; and
R^{d} and R^{e} are each, independently of one another, chosen from the group consisting of alkyl groups, alkenyl groups, alkynyl groups, cycloalkenyl groups, cycloalkynyl groups, and aryl groups optionally condensed with a heterocycle, alkaryl, arylalkyl or heteroaryl;
or R^{d} and R^{e} form, together, a hydrocarbon-based chain containing from 2 to 4 carbon atoms, optionally interrupted with a group chosen from —O—, —S—and —NR^{h}—; where R^{h} is chosen from the group consisting of alkyl groups, alkenyl groups, alkynyl groups, cycloalkenyl groups, cycloalkynyl groups, and aryl groups optionally condensed with a heterocycle, alkaryl, arylalkyl or heteroaryl;
(iii) an —NRⁱ R^{j} group, in which:
Rⁱ and R^{j} are each, independently of one another, chosen from the group consisting of radicals chosen from alkyl groups, haloalkyl groups, alkenyl groups, alkynyl groups, acyl groups, ester groups, aryl groups, arylalkyl groups, arylalkenyl groups, arylalkynyl groups, hydrocarbon-based rings and heterocycles; or
Rⁱ and R^{j} form, together, a hydrocarbon-based chain containing from 2 to 4 carbon atoms, optionally interrupted with an —O—, —S—, or —NR^{h}— group, where R^{h} is as defined above;
R^{2a} a represents a group chosen from the group consisting of hydrogen atoms, halogen atoms, alkyl groups, haloalkyl groups, acyl groups, aryl groups, arylalkyl groups, hydrocarbon-based rings, heterocycles, polymer chains and groups chosen from the group consisting of —CH₂)ₘ—OR^{k}, —CH(OR^{k})(OR^{l}), —(CH₂)ₘ—SR^{k}, —(CH₂)ₘ—S(O)R^{k}, —(CH₂)ₘ—SO₂R ^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR^{l}), (CH₂)ₘ—SiR^{k}R^{l}R^{m}, —(CH₂)ₘ—COOR^{k}, —(CH₂)ₘ—NCOR^{k}and —(CH₂)ₘ—NR^{k}R^{l}, in which R^{k}, R^{l}, R^{m} and m are as defined above,
R¹ and n are as defined above,
wherein the method comprises at least stages a-e:
a—reacting said compound of general formula (IIA) with at least one olefin of general formula (A) 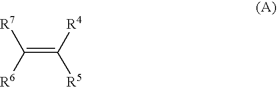
in which:
R⁴, R⁵, R⁶ and R⁷ are as defined above, with at least one of the R⁴, R⁵, R⁶ or R⁷ groups representing a hydrogen atom,
so as to obtain at least one compound of general formula (IIIA) 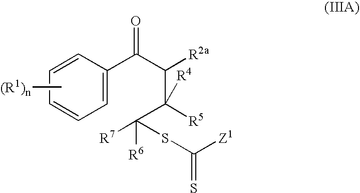
in which:
R¹, R^{2a}, R⁴, R⁵, R⁶, R⁷, Z^{l} and n are as defined above,
b—cyclizing, by radical-based process, said compound of general formula (IIIA) so as to obtain at least one tetralone compound of general formula (IVA) 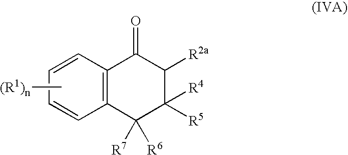
in which:
R¹, R^{2a}, R⁴, R⁵, R⁶, R⁷and n are as defined above,
c—converting said compound of general formula (IVA) into at least its oxime derivative of general formula (VA) 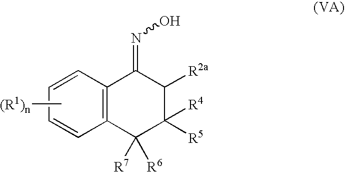
in which:
R¹, R^{2a}, R⁴, R⁵, R⁶, R⁷and n are as defined above,
d—converting said compound of general formula (VA), by Beckmann rearrangement and consecutive reduction(s), into at least one compound of general formula (IA), and
e—recovering said compound of general formula (IA).

53. The method according to claim 52, wherein, in general formula (IA), n =1.

54. The method according to claim 53, wherein the R¹ group is in a para-position.

55. The method according to claim 52, wherein, in general formula (IA), R¹ is chosen from the group consisting of halogen atoms and alkoxy groups.

56. The method according to claim 52, wherein, in general formula (IA), R² and R³ are each independently chosen from the group consisting of hydrogen atom and alkyl groups.

57. The method according to claim 52, wherein, in general formula (IA), R² and R³ are each chosen from the group consisting of halogen atoms.

58. The method according to claim 52, wherein, in the compound of formula (IIA), Z^{l} represents —OR^{a}.

59. The method according to claim 52, wherein the olefin of general formula (A) is disubstituted.

60. The method according to claim 52, wherein the olefin of general formula (A) is monosubstituted.

61. The method according to claim 52, wherein the substituent(s) of said olefin of general formula (A) is (are) chosen from —Oacyl groups and groups of —(CH₂)ₚCN type with p representing an integer ranging from 1 to 10.

62. The method according to claim 52, wherein the olefin of formula (A) is chosen from:
vinyl pivalate,
allyl cyanide, and
N-vinylphthalimide.

63. The method according to claim 52, wherein stage a is carried out in the presence of an effective amount of at least one radical initiator.

64. The method according to claim 52, wherein stage b is carried out in an acidic medium.

65. A method of preparing at least one compound of general formula (IB) 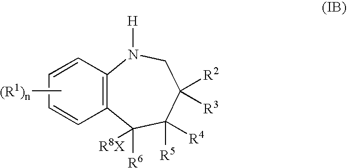
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, and:
R^{k}, R¹ and R^{m} are each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, and heterocycles,
or R^{k} and R¹ form, together with the atom to which they are attached, a heterocycle,
with m denoting an integer greater than or equal to 0,
n represents an integer chosen from the group consisting of 0, 1, 2, 3 and 4, with, when n is greater than or equal to 2, r the corresponding R¹ groups are identical or different, and form, together, a hydrocarbon-based ring or a heterocycle,
R², R³, R⁴, R⁵ and R⁶, independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituents groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, with R^{k}, R¹, R^{m} and m as defined above,
or R⁴, R⁵ and R⁶ form, in pairs, one or more hydrocarbon-based ring(s) or heterocycle(s), with at least one of the R⁴, R⁵ and R⁶ groups representing a hydrogen atom,
X is chosen from the group consisting of O, NR⁹, S, S(O), SO₂, SO₂NR⁹, and R⁸ and R⁹ are each, independently of one another, chosen from the group consisting of hydrogen atom, alkyl groups, haloalkyl groups, alkenyl groups, alkynyl groups, acyl groups, aryl groups, arylalkyl groups, alkaryl groups, arylalkenyl groups, arylalkynyl groups, hydrocarbon-based rings, heterocycles, and polymer chains, optionally substituted,
or R⁸ and R⁹ form, together with the atom to which they are attached, a heterocycle from at least one compound of general formula (IVB) 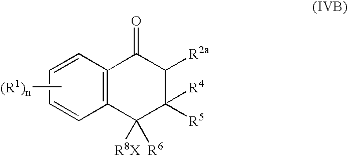
in which:
R¹, R⁴, R⁵, R⁶, R⁸, X and n are as defined above, and
R^{2a} represents a group chosen from the group consisting of hydrogen atom, halogen atoms, alkyl groups, haloalkyl groups, acyl groups, aryl groups, arylalkyl groups, hydrocarbon-based rings, heterocycles, polymer chains and groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), (CH₂)ₘ—SiR^{k}R¹R^{m}, —(CH₂)ₘ—COOR^{k}, —(CH₂)ₘ—NCOR^{k} and —(CH₂)ₘ—NR^{k}R¹, in which R^{k}, R¹, R^{m} and m are as defined above,
wherein the method comprises at least stages a′-c′:
a′—converting said compound of general formula (IVB) into at least its oxime derivative of general formula (VB) 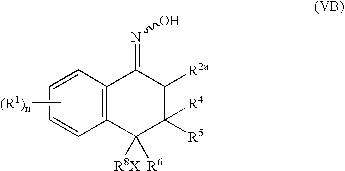
in which:
R¹, R^{2a}, R⁴, R⁵, R⁶, R⁸, X and n are as defined above,
b′—converting said compound of general formula (VB), by Beckmann rearrangement and consecutive reduction(s), into at least said compound of general formula (IB), and
c′—recovering said compound of general formula (IB).

66. The method according to claim 52, wherein the stage c consisting of preparation of the oxime derivative of formula (VA) comprises placing said compound of general formula (IVA) in the presence of an effective amount of nitromethane or of hydroxylamine.

67. The method according to claim 66, further comprising a stage consisting of recovery of the product of formula (VA).

68. The method according to claim 52, wherein the conversion of the compound (VA) by Beckmann rearrangement is carried out in the presence of an effective amount of PCl₅.

69. The method according to claim 68, wherein the PCl₅ is used in molar excess relative to the compound of formula (VA).

70. The method according to claim 52, wherein the product derived from the Beckmann rearrangement is reduced with an effective amount of at least one metal reducing agent.

71. The method according to claim 70, wherein the reduction product obtained is treated with an effective amount of reducing agent.

72. A compound of general formula (IA) 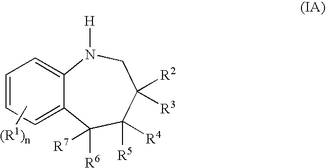
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, and:
R^{k}, R¹ and R^{m} are each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, and heterocycles,
or R^{k} and R¹ form, together with the atom to which they are attached, a heterocycle,
with m denoting an integer greater than or equal to 0,
R², R³, R⁴, R⁵ and R⁶ independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘOR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, with R^{k}, R¹, R^{m} and m as defined above,
or R⁴, R⁵ and R⁶ form, in pairs, one or more hydrocarbon-based ring(s) or heterocycle(s), with at least one of the R⁴, R⁵ and R⁶ groups representing a hydrogen atom,
R⁷=—XR⁸, where X is chosen from the group consisting of O, NR⁹, S, S(O), SO₂, SO₂NR⁹, and R⁸ and R⁹ are each, independently of one another, chosen from the group consisting of hydrogen atom, alkyl groups, haloalkyl groups, alkenyl groups, alkynyl groups, acyl groups, aryl groups, arylalkyl groups, alkaryl groups, arylalkenyl groups, arylalkynyl groups, hydrocarbon-based rings, heterocycles, and polymer chain, optionally substituted,
or R⁸ and R⁹ form, together with the atom to which they are attached, a heterocycle from at least one compound of general formula (IVB) 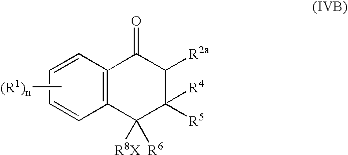
in which:
R¹, R⁴, R⁵, R⁶, R⁸, X and n are as defined above, and
R^{2a} represents a group chosen from the group consisting of hydrogen atom, halogen atoms, alkyl groups, haloalkyl groups, acyl groups, aryl groups, arylalkyl groups, hydrocarbon-based rings, heterocycles, polymer chains and groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), (CH₂)ₘ—SiR^{k}R¹R^{m}, —(CH₂)ₘ—COOR^{k}, —(CH₂)ₘ—NCOR^{k} and —(CH₂)ₘ—NR^{k}R¹, in which R^{k}, R¹, R^{m} and m are as defined above, and n=1.

73. The compound according to claim 72, wherein the compound is chosen from the group consisting of:
7-chloro-2,3,4,5-tetrahydro-1H-benzo[b]azepin-5-yl 2,2-dimethylpropionate,
7-fluoro-2,3,4,5-tetrahydro-1H-benzo[b]azepin-5-yl 2,2-dimethylpropionate,
7-methoxy-2,3,4,5-tetrahydro-1H-benzoazepin-5-yl 2,2-dimethylpropionate,
(7-fluoro-2,3,4,5-tetrahydro-1H-benzo[b]azepin-5-yl)acetonitrile,
3,3,7-tricholoro-2,3,4,5-tetrahydro-1H-benzo[b]azepin-5-yl 2,2-dimethylpropionate, and
derivatives thereof.

74. A compound of general formula (VB) 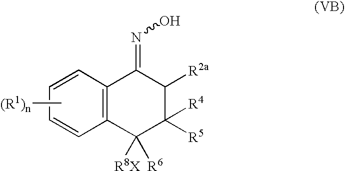
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k} and —(CH₂)ₘ—NR^{k}R¹, and:
R^{k}, R¹ and R^{m} each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, or R^{k} and R¹ form, together with the atom to which they are attached, a heterocycle, with m denoting an integer greater than or equal to 0,
R⁴, R⁵ and R⁶ independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituents groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, with R^{k}, R¹, R^{m} and m as defined above, or R⁴, R⁵ and R⁶ form, in pairs, one or more hydrocarbon-based ring(s) or heterocycle(s), with at least one of the R⁴, R⁵ and R⁶ groups representing a hydrogen atom,
R^{2a} represents a group chosen from the group consisting of hydrogen atoms, halogen atoms, alkyl groups, haloalkyl groups, acyl groups, aryl groups, arylalkyl groups, hydrocarbon-based rings, heterocycles, polymer chains and groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), (CH₂)ₘ—SiR^{k}R¹R^{m}, —(CH₂)ₘ—COOR^{k}, —(CH₂)ₘ—NCOR^{k} and —(CH₂)ₘ—NR^{k}R¹, in which R^{k}, R¹, R^{m} and m are as defined above,
X is chosen from the group consisting of O, NR⁹, S, S(O), SO₂, SO₂NR⁹, and R⁸ and R⁹ are each, independently of one another, chosen from the group consisting of hydrogen atoms, alkyl groups, haloalkyl groups, alkenyl groups, alkynyl groups, acyl groups, aryl groups, arylalkyl groups, alkaryl groups, arylalkenyl groups, arylalkynyl groups, hydrocarbon-based rings, heterocycles, and polymer chains, optionally substituted,
or R⁸ and R⁹ form, together with the atom to which they are attached, a heterocycle from at least one compound of general formula (IVB) 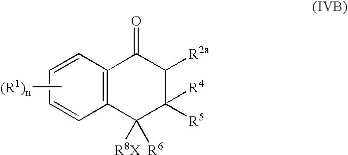
in which:
R¹, R^{2a}, R⁴, R⁵, R⁶, R⁸, X and n are as defined above.

75. A compound according to claim 74, wherein the compound is chosen from the group consisting of:
4-[(E)-hydroxyimino]-7-chloro-1,2,3,4-tetrahydronaphthalen-1-yl 2,2-dimethylpropionate,
4-[(E)-hydroxyimino]-7-fluoro-1,2,3,4-tetrahydronaphthalen-1-yl 2,2-dimethylpropionate, and
4-[(E)-hydroxyimino]-7-methoxy-1,2,3,4-tetrahydronaphthalen-1-yl 2,2-dimethylpropionate, and
derivatives thereof.

76. A method of preparing a benzazepine of general formula (VIA): 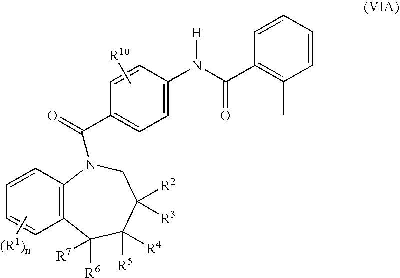
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, and:
R^{k}, R¹ and R^{m} are each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, and heterocycles,
or R^{k} and R¹ form, together with the atom to which they are attached, a heterocycle,
with m denoting an integer greater than or equal to 0,
R², R³, R⁴, R⁵ and R⁶ independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, (CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, with R^{k}, R¹, R^{m} and m as defined above,
or R⁴, R⁵ and R⁶ form, in pairs, one or more hydrocarbon-based ring(s) or heterocycle(s), with at least one of the R⁴, R⁵ and R⁶ groups representing a hydrogen atom,
R⁷=—XR⁸, where X is chosen from the group consisting of O, NR⁹, S, S(O), SO₂, SO₂NR⁹, and R⁸ and R⁹ are each, independently of one another, chosen from the group consisting of hydrogen atom, alkyl groups, haloalkyl groups, alkenyl groups, alkynyl groups, acyl groups, aryl groups, arylalkyl groups, alkaryl groups, arylalkenyl groups, arylalkynyl groups, hydrocarbon-based rings, heterocycles, and polymer chain, optionally substituted,
or R⁸ and R⁹ form, together with the atom to which they are attached, a heterocycle from at least one compound of general formula (IVB) 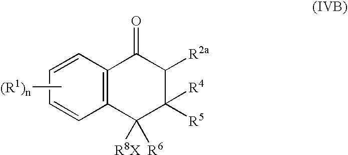
in which:
R¹, R⁴, R⁵, R⁶, R⁸, X and n are as defined above, and
R^{2a} represents a group chosen from the group consisting of hydrogen atom, halogen atoms, alkyl groups, haloalkyl groups, acyl groups, aryl groups, arylalkyl groups, hydrocarbon-based rings, heterocycles, polymer chains and groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), (CH₂)ₘ—SiR^{k}R¹R^{m}, —(CH₂)ₘ—COOR^{k}, —(CH₂)ₘ—NCOR^{k} and —(CH₂)ₘ—NR^{k}R¹, in which R^{k}, R¹, R^{m} and m are as defined above,
n=1, and
R¹⁰ is chosen from the group consisting of hydrogen atom, alkyl groups and acyl groups,
wherein the method comprises converting a compound of general formula (IIA) into a compound of formula (IA) according to the method according to claim 52.

77. A method of preparing a benzazepine of general formula (VIB): 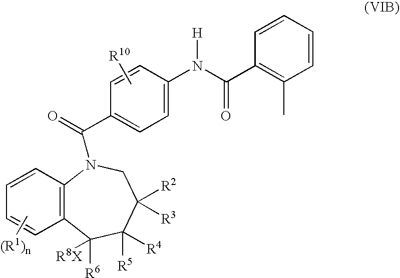
in which:
R¹ is chosen from the group consisting of halogen atoms chosen from the group consisting of chlorine, fluorine, bromine and iodine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ13 SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, and:
R^{k}, R¹ and R^{m} are each independently chosen from the group consisting of hydrogen atom, alkyl groups, haloalkyl groups, alkenyl groups, alkynyl groups, acyl groups, aryl groups, arylalkyl groups, arylalkenyl groups, arylalkynyl groups, hydrocarbon-based rings, and heterocycles,
or R^{k} and R¹ form, together with the atom to which they are attached, a heterocycle,
with m denoting an integer greater than or equal to 0,
R², R³, R⁴, R⁵ and R⁶ independently of one another, are chosen from the group consisting of hydrogen atom, halogen atoms chosen from the group consisting of chlorine, fluorine and bromine, alkyl groups, haloalkyl groups, alkenyl groups, alkynyl groups, acyl groups, aryl groups, arylalkyl groups, arylalkenyl groups, arylalkynyl groups, hydrocarbon-based rings, heterocycles, polymer chains, and substituent groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), —(CH₂)ₘ—SiR^{k}R¹R^{m}, —(CH₂)ₘ—COOR^{k}, —(CH₂)ₘ—NCOR^{k}, and —(CH₂)ₘ—NR^{k}R¹, with R^{k}, R¹, R^{m} and m as defined above,
or R⁴, R⁵ and R⁶ form, in pairs, one or more hydrocarbon-based ring(s) or heterocycle(s), with at least one of the R⁴, R⁵ and R⁶ groups representing a hydrogen atom,
R⁷=—XR⁸, where X is chosen from the group consisting of O, NR⁹, S, S(O), SO₂, SO₂NR⁹, and R⁸ and R⁹ are each, independently of one another, chosen from the group consisting of hydrogen atom, alkyl groups, haloalkyl groups, alkenyl groups, alkynyl groups, acyl groups, aryl groups, arylalkyl groups, alkaryl groups, arylalkenyl groups, arylalkynyl groups, hydrocarbon-based rings, heterocycles, and polymer chain, optionally substituted,
or R⁸ and R⁹ form, together with the atom to which they are attached, a heterocycle from at least one compound of general formula (IVB) 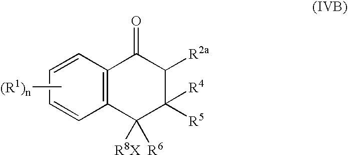
in which:
R¹, R⁴, R⁵, R⁶, R⁸, X and n are as defined above, and
R^{2a} represents a group chosen from the group consisting of hydrogen atom, halogen atoms, alkyl groups, haloalkyl groups, acyl groups, aryl groups, arylalkyl groups, hydrocarbon-based rings, heterocycles, polymer chains and groups chosen from the group consisting of —(CH₂)ₘ—OR^{k}, —CH(OR^{k})(OR¹), —(CH₂)ₘ—SR^{k}, —(CH₂)ₘ—S(O)R^{k}, —(CH₂)ₘ—SO₂R^{k}, —(CH₂)ₘ—SO₂NR^{k}R¹, —(CH₂)ₘ—SO₃R^{k}, —(CH₂)ₘ—NO₂, —(CH₂)ₘ—CN, —(CH₂)ₘ—PO(OR^{k})(OR¹), (CH₂)ₘ—SiR^{k}R¹R^{m}, —(CH₂)ₘ—COOR^{k}, —(CH₂)ₘ—NCOR^{k} and —(CH₂)ₘ—NR^{k}R¹, in which R^{k}, R¹, R^{m} and m are as defined above,
n=1, and
R¹⁰ is chosen from the group consisting of hydrogen atom, alkyl groups and acyl groups,
wherein the method comprises converting a compound of general formula (IVB) into a compound of formula (IB) according to the method according to claim 65.

78. The method according to claim 52, wherein, in general formula (IIA), R^{2a} is a hydrogen atom.

79. The method according to claim 52, wherein, in general formula (IIA), R^{2a} is a halogen atom chosen from the group consisting of chlorine atoms, fluorine atoms and bromine atoms.

80. The method according to claim 57, wherein, in general formula (IA), R² and R³ are each chosen from the group consisting of chlorine atoms, fluorine atoms and bromine atoms.

81. The method according to claim 58, wherein R^{a} is chosen from the group consisting of C₁ to C₁₂ alkyl groups.

82. The method according to claim 59, wherein the olefin of general formula (A) is terminal disubstituted or cyclic.

83. The method according to claim 60, wherein, in the olefin of general formula (A), R⁴, R⁵ and R⁶ are each hydrogen atoms.

84. The method according to claim 63, wherein said at least one radical initiator is dilauroyl peroxide (DLP).

85. The method according to claim 64, wherein the acidic medium is camphorsulfonic acid.

86. The method according to claim 67, further comprising recovery of the product of formula (VA) is accomplished by recrystallization.

87. The method according to claim 65, wherein the stage a′ consisting of preparation of the oxime derivative of formula (VB) comprises placing said compound of general formula (IVB) in the presence of an effective amount of nitromethane or of hydroxylamine.

88. The method according to claim 87, further comprising a stage consisting of recovery of the product of formula (VB).

89. The method according to claim 88, further comprising recovery of the product of formula (VB) is accomplished by recrystallization.

90. The method according to claim 65, wherein the conversion of the compound (VB) by Beckmann rearrangement is carried out in the presence of an effective amount of PCl₅.

91. The method according to claim 90, wherein the PCl₅ is used in molar excess relative to the compound of formula (VB).

92. The method according to claim 65, wherein the product derived from the Beckmann rearrangement is reduced with an effective amount of at least one metal reducing agent.

93. The method according to claim 92, wherein the reduction product obtained is treated with an effective amount of reducing agent.

94. The method according to claim 76, wherein R¹⁰ represents a methyl group.

95. The method according to claim 77, wherein R¹⁰ represents a methyl group.
